(19) United States
(12) Patent Application Publication
(10) Pub. No.: US 2024/0100001 A1
(43) Pub. Date: Mar. 28, 2024

(54) DENATONIUM SALT FOR USE IN PREVENTING, PREVENTING PROGRESSION AND TREATING FATTY LIVER DISEASES

(71) Applicant: AARDVARK THERAPEUTICS INC., San Diego, CA (US)

(72) Inventors: Zhenhuan ZHENG, San Diego, CA (US); Andreas NIETHAMMER, San Diego, CA (US); Anjuli TIMMER, San Diego, CA (US); Tien-Li LEE, San Diego, CA (US)

(73) Assignee: AARDVARK THERAPEUTICS INC., San Diego, CA (US)

(21) Appl. No.: 18/274,180

(22) PCT Filed: Jan. 31, 2022
(86) PCT No.: PCT/US2022/014550

§ 371 Date: Jul. 25, 2023

Related U.S. Application Data
(60) Provisional application No. 63/144,386, filed on Feb. 1, 2021.

(51) Int. Cl. A61K 31/167; A61P 1/16

(52) U.S. Cl. CPC A61K 31/167; A61P 1/16

(57) ABSTRACT

There is disclosed a pharmaceutical composition comprising a bitter receptor agonist comprising a denatonium salt, wherein the denatonium salt is selected from the group consisting of denatonium acetate (DA), denatonium citrate, denatonium maleate, denatonium saccharide, and denatonium tartrate, for use in a method for preventing, preventing progression and/or treating a fatty liver disease. In some embodiments, the daily dose of the denatonium salt for preventing or preventing progression of a fatty liver disease is from 25 mg/kg to 45 mg/kg QD and the daily dose of the denatonium salt for treating an existing fatty liver disease is from 70 mg/kg to 200 mg/kg per day administered QD or BID.

### CROSS-REFERENCE TO RELATED APPLICATIONS

This application claims the benefit of priority of U.S. Provisional Patent Application No. 63/144,386, filed Feb. 1, 2021.

### TECHNICAL FIELD

The present disclosure provides a method for preventing, preventing progression and treating a fatty liver disease, comprising administering an effective amount of a pharmaceutical composition comprising a bitter receptor agonist comprising a denatonium salt, wherein the denatonium salt is selected from the group consisting of denatonium acetate (DA), denatonium citrate, denatonium maleate, denatonium saccharide, and denatonium tartrate; and wherein the daily dose of the denatonium salt for preventing or preventing progression of a fatty liver disease is from about 0.1 mg/kg/day to about 8 mg/kg/day and the daily dose of the denatonium salt for treating an existing fatty liver disease is from about 1 mg/kg/day to about 8 mg/kg/day, each administered QD, BID or TID.

### BACKGROUND

Fatty liver disease is a term to describe a group of liver diseases including nonalcoholic steatohepatitis (NASH), alcoholic steatohepatitis (ASH), non-alcoholic fatty liver disease (NAFLD), and HIV-associated steatohepatitis, with or without liver fibrosis. Specifically, nonalcoholic steatohepatitis (NASH) is a common liver disease that is associated with increased morbidity and mortality. But there are no FDA-approved treatment options despite many compounds being tested in what are purported to be NASH treatment models. Non-alcoholic fatty liver disease (NAFLD) is a disorder affecting as many as 1 in 3-5 adults and 1 in 10 children in the United States. These are conditions where there is an accumulation of excess fat in the liver of people who drink little or no alcohol. The most common form of NAFLD is a non-serious condition called hepatic steatosis (fatty liver), in which fat accumulates in the liver cells: although this is not normal, by itself it probably does not damage the liver. NAFLD most often presents itself in individuals with a constellation of risk factors called the metabolic syndrome, which is characterized by elevated fasting plasma glucose (FPG) with or without intolerance to post-prandial glucose, being overweight or obese, high blood lipids such as cholesterol and triglycerides (TGs) and low high-density lipoprotein cholesterol (HDL-C) levels, and high blood pressure; but not all patients have all the manifestations of the metabolic syndrome. Obesity is thought to be the most common cause of NAFLD; and some experts estimate that about two-thirds of obese adults and one-half of obese children may have fatty liver. The majority of individuals with NAFLD have no symptoms and a normal physical examination (although the liver may be slightly enlarged); children may exhibit symptoms such as abdominal pain and fatigue, and may show patchy dark skin discoloration (acanthosis nigricans). The diagnosis of NAFLD is usually first suspected in an overweight or obese person who is found to have mild elevations in their liver blood tests during routine testing, though NAFLD can be present with normal liver blood tests, or incidentally detected on imaging investigations such as abdominal ultrasound or CT scan. It is confirmed by imaging studies, most commonly a liver ultrasound or magnetic resonance imaging (MRI), and exclusion of other causes.

Some people with NAFLD may develop a more serious condition called non-alcoholic steatohepatitis (NASH): about 2-5% of adult Americans and up to 20% of those who are obese may suffer from NASH. In NASH, fat accumulation in the liver is associated with inflammation and different degrees of scarring. NASH is a potentially serious condition that carries a substantial risk of progression to end-stage liver disease, cirrhosis and hepatocellular carcinoma. Some patients who develop cirrhosis are at risk of liver failure and may eventually require a liver transplant. Therefore, weight loss is a recommended means to prevent NASH or slow the progression of NASH. However, weight loss has not been shown to treat NASH once the liver fibrosis damage has occurred.

NAFLD may be differentiated from NASH by the NAFLD Activity Score (NAS), the sum of the histopathology scores of a liver biopsy for steatosis (0 to 3), lobular inflammation (0 to 2), and hepatocellular ballooning (0 to 2). A NAS of <3 corresponds to NAFLD, 3-4 corresponds to borderline NASH, and ≥5 corresponds to NASH. The biopsy is also scored for fibrosis (0 to 4).

NASH is a leading cause of end-stage liver disease.

### Treatments for NAFLD and NASH

There are no drugs currently approved to prevent or treat NAFLD or NASH. A number of pharmacological interventions have been tried in NAFLD/NASH but with overall limited benefit. Antioxidant agents may arrest lipid peroxidation and cytoprotective agents stabilize phospholipid membranes, but agents tried unsuccessfully or with only modest benefit so far include ursodeoxycholic acid, vitamins E (α-tocopherol) and C, and pentoxifylline. Weight-loss agents such as orlistat, have had no significant benefit compared to just the use of diet and exercise to achieve weight loss (“weight loss alone”). Many weight-loss studies in NAFLD/NASH have been pilot studies of short duration and limited success, reporting only a modest improvement in necroinflammation or fibrosis. A randomized, double-blind, placebo-controlled 6-month trial (Belfort, “A placebo-controlled trial of pioglitazone in subjects with nonalcoholic steatohepatitis”, N. Engl. J. Med., 355, 2297-2307 (2006)) of weight loss alone against pioglitazone, a thiazolidinedione peroxisome proliferator-activated receptor-γ (PPARγ) agonist and insulin sensitizer, failed to demonstrate any improvement for weight loss alone, but treatment with pioglitazone improved glycemic control, insulin sensitivity, indicators of systemic inflammation (including hsCRP, tumor necrosis factor-α, and transforming growth factor-β), and liver histology in patients with NASH and IGT or T2DM. Treatment with pioglitazone also ameliorated adipose, hepatic, and muscle IR, and was associated with an approximately 50% decrease in necroinflammation (p<0.002) and a 37% reduction in fibrosis (p=0.08). Improvement in hepatocellular injury and fibrosis has been reported in another controlled trial with pioglitazone of 12 months duration. In contrast, while the first randomized clinical study with rosiglitazone, the other thiazolidinedione approved for diabetes treatment, in NASH demonstrated a reduction in IR, plasma alanine aminotransferase (ALT) levels and steatosis, rosiglitazone treatment had no significant effect on necrosis, inflammation, or fibrosis. It is important to note with these results that even reduced ALT, insulin resistance and other diabetes indicators did not decreases liver fibrosis, which is a key indicator of NASH. Therefore, controlling diabetes is not enough to treat NASH or even prevent NASH. Moreover, there are severe safety limitations with both pioglitazone and Rosiglitazone. A preliminary report of the 2-year, open-label follow-up of this trial was also disappointing, with no significant benefit from rosiglitazone treatment. One pharmacological agent with some efficacy in NASH is pioglitazone. Unfortunately, pioglitazone is also associated with a significantly increased risk of weight gain, edema, congestive heart failure, and osteoporotic fractures in both women and men.

A phase 2 trial involving patients with NASH showed that treatment with daily subcutaneously-administered semaglutide (GLP-1 agonist) resulted in a higher percentage of patients with NASH resolution than placebo. However, the trial did not show a significant between-group difference in the percentage of patients with an improvement in fibrosis stage (Newsome et al., N. Engl. J. Med. “A Placebo-Controlled Trial of Subcutaneous Semaglutide in Nonalcoholic Steatohepatitis” Nov. 13, 2020). Unfortunately, “The percentage of patients in whom NASH resolution was achieved with no worsening of fibrosis was 40% in the 0.1-mg group, 36% in the 0.2-mg group, 59% in the 0.4-mg group, and 17% in the placebo group (P=0.48). The mean percent weight loss was 13% in the 0.4-mg group and 1% in the placebo group. The incidence of nausea, constipation, and vomiting was higher in the 0.4-mg group than in the placebo group (nausea, 42% vs. 11%; constipation, 22% vs. 12%; and vomiting, 15% vs. 2%). Malignant neoplasms were reported in 3 patients who received semaglutide (1%) and in no patients who received placebo. Overall, neoplasms (benign, malignant, or unspecified) were reported in 15% of the patients in the semaglutide groups and in 8% in the placebo group; no pattern of occurrence in specific organs was observed.” Accordingly, even GLP-1 agonists, such as semaglutide, are not benign treatments for NASH prevention or treatment to warrant the risk of long-term administration needed to treat, prevent or slow progression of NASH.

A summary of the clinical data obtained indicates that treatment of NASH seems to be uncoupled from weight loss as a treatment means, by any weight loss technique. Although weight loss may be an effective means for prevention of NASH or possibly showing progression of NASH. Therefore, there is a need for better accepted translational models to predict prevention, preventing progression and treatment of fatty liver diseases, including NASH. Therefore, there is a need for effective and safer NASH treatment options, particularly if a treatment can be delivered orally and not by injection. There is also a need for safe agents to prevent development of full NASH liver disease and damage and to show progression of NASH.

### SUMMARY

The present disclosure provides a method for preventing, preventing progression and/or treating a fatty liver disease, comprising administering an effective amount of a pharmaceutical composition comprising a bitter receptor agonist comprising a denatonium salt, wherein the denatonium salt is selected from the group consisting of denatonium acetate (DA), denatonium citrate, denatonium maleate, denatonium saccharide, and denatonium tartrate; and wherein the daily dose of the denatonium salt for preventing or preventing progression of a fatty liver disease is from about 0.1 mg/kg/day to about 8 mg/kg/day and the daily dose of the denatonium salt for treating an existing fatty liver disease is from about 1 mg/kg/day to about 8 mg/kg/day, each administered QD, BID, or TID.

More specifically, the present disclosure provides a method for preventing and preventing progression of fatty liver diseases selected from the group consisting of nonalcoholic steatohepatitis (NASH), alcoholic steatohepatitis (ASH), non-alcoholic fatty liver disease (NAFLD), and HIV-associated steatohepatitis, with or without liver fibrosis, comprising administering an effective amount of a pharmaceutical composition comprising a comprising a denatonium salt, wherein the denatonium salt is selected from the group consisting of denatonium acetate (DA), denatonium citrate, denatonium maleate, denatonium saccharide, and denatonium tartrate and wherein the daily dose of the denatonium salt for preventing or preventing progression of a fatty liver disease is from about 0.1 mg/kg/day to about 8 mg/kg/day QD, BID, or TID. Preferably, the pharmaceutical composition further comprises from about 0.5 g to about 5 g acetic acid. More preferably, the dosage per day of the acetic acid for an adult is from about 1.5 g to about 3 g.

The present disclosure provides a method for treating liver diseases selected from the group consisting of nonalcoholic steatohepatitis (NASH), alcoholic steatohepatitis (ASH), non-alcoholic fatty liver disease (NAFLD), and HIV-associated steatohepatitis, with or without liver fibrosis, comprising administering an effective amount of a pharmaceutical composition comprising a denatonium salt, wherein the denatonium salt is selected from the group consisting of denatonium acetate (DA), denatonium citrate, denatonium maleate, denatonium saccharide, and denatonium tartrate, and wherein the daily dose of the denatonium salt for treating an existing fatty liver disease is from about 1.0 mg/kg/day to about 8 mg/kg per day administered QD, BID, or TID. Preferably, the pharmaceutical composition further comprises from about 0.5 g to about 5 g acetic acid. More preferably, the dosage per day of the acetic acid for an adult is from about 1.5 g to about 3 g.

### BRIEF DESCRIPTION OF THE FIGURES

FIGS. 1A-B show group mean absolute (FIG. 1A) and relative (% baseline; FIG. 1B) body weights of AMLN diet-fed mice during one year of daily treatment with vehicle or 30 mg/kg DA.

FIG. 2 shows fasting blood glucose levels in the DA-treated mice were significantly lower than those in vehicle-treated mice at Week 24 only. Comparison to vehicle value on respective day by two-tailed non-paired t-test. A difference was considered statistically significant with p<0.05. *p<0.05. NS, not significant.

FIG. 3 shows insulin levels in the DA-treated mice were significantly elevated (compared to those in vehicle-treated mice) at Weeks 4 and 12, but not subsequently. Comparison to vehicle value on respective day by two-tailed non-paired t-test. A difference was considered statistically significant with p<0.05. *p<0.05. **p<0.01. NS, not significant.

FIG. 4 shows HbA1c levels in the DA-treated mice were significantly attenuated (compared to those in vehicle-treated mice) at Weeks 12 and 48, though not at Week 24. (HbA1c levels were not assayed pre-dose or at Week 4.) Comparison to vehicle value on respective day by two-tailed non-paired t-test. A difference was considered statistically significant with p<0.05. *p<0.05. **p<0.01. ***p<0.001. NS, not significant. HbA1c not assayed pre-dose or at Week 4.

FIG. 5A shows GLP-1 levels in the DA-treated mice were significantly attenuated (compared to those in vehicle-treated mice) only at Week 24. While the magnitude of the difference was similar to that at other time points (which lack statistical significance), the variance at Week 24 was lower than at other weeks, suggesting that the statistical significance this effect was not meaningful. Comparison to vehicle value on respective day by two-tailed non-paired t-test. A difference was considered statistically significant with p<0.05. ***p<0.001. NS, not significant. FIG. 5B shows GLP-2 levels. Note that GLP-2 was assayed only at Week 48. GLP-2 levels in the DA-treated mice did not differ significantly from those in vehicle-treated mice at Week 48, the only time point for which this parameter was assessed.

FIG. 6 shows Serum ALT activity levels in the DA-treated mice were significantly attenuated (compared to those in vehicle-treated mice) at Weeks 24 and 48, but not earlier. Comparison to vehicle value on respective day by two-tailed non-paired t-test. A difference was considered statistically significant with p<0.05. **p<0.01. ***p<0.001. NS, not significant.

FIG. 7 shows Serum AST activity levels in the DA-treated mice were significantly attenuated (compared to those in vehicle-treated mice) only at Week 24. Comparison to vehicle value on respective day by two-tailed non-paired t-test. A difference was considered statistically significant with p<0.05. **p<0.01. NS, not significant.

FIG. 8 shows Serum ALB levels in the DA-treated mice did not differ significantly from those in vehicle-treated mice at any assessed time point. Comparison to vehicle value on respective day by two-tailed non-paired t-test. A difference was considered statistically significant with p<0.05. NS, not significant.

FIG. 9 shows Serum BA levels in the DA-treated mice were significantly attenuated (compared to those in vehicle-treated mice) at Week 24 only. Comparison to vehicle value on respective day by two-tailed non-paired t-test. A difference was considered statistically significant with p<0.05. *p<0.05. NS, not significant.

FIG. 10 shows Blood levels of cytokines (n=10) that showed statistically significant differences in AMLN diet-fed mice during one year of daily treatment with vehicle or 30 mg/kg DA for IL-6 (FIG. 10A), IL-9 (FIG. 10B), IL-13 (FIG. 10C), IP-10 (FIG. 10D), KC (FIG. 10E), MCP-1 (FIG. 10F), MIP-1a (FIG. 10G), MIP-1B (FIG. 10H), MIG (FIG. 10I), and TNFα-6 (FIG. 10J). Parenthetical integers in cytokine names/graph titles correspond to assay numbers used as part of the multiplex assay. Comparison to vehicle value on respective day by two-tailed non-paired t-test. A difference was considered statistically significant with p<0.05. *p<0.05. **p<0.01. NS, not significant.

FIGS. 11A (absolute) and B relative (body-weight-normalized) show necropsy liver weight after one year of daily treatment with vehicle of 30 mg/kg DA. Comparison to vehicle value on respective day by two-tailed non-paired t-test. A difference was considered statistically significant with p<0.05. *p<0.05. NS, not significant.

FIG. 12 shows serum HDL, LDL, and TGA levels after one year of daily treatment with vehicle or 30 mg/kg DA. Serum TGA levels, but not those of HDL or LDL, were significantly attenuated in the DA-treated mice (compared to vehicle-dosed animals) after one year of daily dosing. DA, denatonium acetate. HDL, high-density lipo-protein. LDL. Low-density lipoprotein. TGA, triglycerides. Comparison to vehicle value on respective day by two-tailed non-paired t-test. A difference was considered statistically significant with p<0.05. *p<0.05. NS, not significant.

FIG. 13 show liver TGA (FIG. 13A), TC (FIG. 13B) and FFA (FIG. 13 C) after one year of daily treatment with vehicle of 30 mg/kg DA. Liver TGA levels, but not those of TC or FFA, were significantly attenuated in the DA-treated mice (compared to vehicle-dosed mice) after one year of daily dosing.

FIG. 14 shows that treatment with DA (ARD-101) significantly improved NAFLD Activity Score based on blinded histopathologic review.

FIGS. 15A and 15B shows that treatment with ARD-101 (DA) showed a remarkable effect on body weight (15A) and body weight change (15B) in AMLN-diet induced NASH mice. Data are presented as means. Statistical analysis was performed with one tailed t-test. ***P<0.001 as compared with vehicle; ^{$$}P<0.01 and ^{$$$}P<0.001 as compared with the combination.

FIGS. 16A and 16B show liver weight (FIG. 16A) and liver/body weight ratio (FIG. 16B) showing that DA (ARD-101) significantly decreased liver weight and liver/body weight ratio as compared to vehicle.

FIG. 17A shows ALT levels and FIG. 17B shows AST levels. At the end of the study, DA (ARD-101) significantly decreased ALT and AST levels as compared to vehicle control.

FIGS. 18A (triglycerides (TG)), 18B (LDL) and 18C (HDL) show that at the end of the study of Example 2, DA (ARD-101) significantly decreased triglycerides (TG), low density lipoproteins (LDL) and high density lipoproteins (HDL), respectively.

FIG. 19 shows the change in glucose level at the end of the study of Example 2 for the indicated treatment groups.

FIG. 20 shows HbA1c levels for the indicated treatment groups at the end of the study of Example 2. The baseline HbA1c level was 5.0%.

FIG. 21 shows the insulin level at the end of the study of Example 2 for the indicated treatment groups. The baseline insulin level was 1.5 ng/ml.

FIG. 22 shows that the two treatments did not significantly impact bile acid levels as compared to vehicle control. The baseline bile acid level was 30 μmol/L.

FIG. 23A (CK-18) shows that DA (ARD-101) significantly decreased CK-18 levels compared to vehicle control. FIG. 23B shows TGF-β1 levels for the indicated treatment groups.

FIGS. 24A and 24B show that at the end of the Example 2 study, the two treatments did not significantly impact IL-6 and TNF-α levels as compared to vehicle.

### DETAILED DESCRIPTION

The present disclosure is based upon a first in vivo study (presented in Example 1) that was focused on fatty liver disease prevention, and then a second in vivo study (presented in Example 2) that was focused on fatty liver disease treatment. Together, these data from both studies provide surprising evidence that treatment with the denatonium salts, at different dosing frequencies and dose ranges, can provide both a fatty liver disease protective effect in susceptible individuals (such as Type 2 diabetics with a much higher risk of developing NASH) and a prevention of disease progression effect at lower, once daily oral dosing of the denatonium salts. And it required a higher and twice daily dosing of the denatonium salts to treat NASH in the in vivo model with similar effects as a GLP-1 agonist but without the known (in humans) severe side effects of semaglutide, a prototype marketed GLP-1 agonist.

### Definitions

A “fatty liver disease” means any of a group of diseases characterized by undesirable accumulation of fat in the liver, including nonalcoholic steatohepatitis (NASH), alcoholic steatohepatitis (ASH), non-alcoholic fatty liver disease (NAFLD), and HIV-associated steatohepatitis, with or without liver fibrosis.

A “therapeutically effective amount” of a denatonium salt means that amount which, when administered to a human for treating a fatty liver disease, such as NAFLD or NASH, is sufficient to effect treatment for the fatty liver disease. “Treating” or “treatment” of NAFLD or NASH in a human includes one or more of
(1) preventing or reducing the risk of developing NAFLD or NASH, i.e., causing the clinical symptoms of NAFLD or NASH not to develop in a subject who may be predisposed to NAFLD or NASH but who does not yet experience or display symptoms of the NAFLD or NASH (i.e. prophylaxis);
(2) inhibiting NAFLD or NASH, i.e., arresting or reducing the development of NAFLD or NASH or its clinical symptoms; and
(3) relieving NAFLD or NASH, i.e., causing regression, reversal, or amelioration of the NAFLD or NASH or reducing the number, frequency, duration or severity of its clinical symptoms. The therapeutically effective amount for a particular subject varies depending upon the health and physical condition of the subject to be treated, the extent of the NAFLD or NASH, the assessment of the medical situation, and other relevant factors. It is expected that the therapeutically effective amount will fall in a relatively broad range that can be determined through routine trial.

“Or” is used in the inclusive sense (equivalent to “and/or”) unless the context requires otherwise.

As used herein, ranges and amounts can be expressed as “about” a particular value or range. About also includes the exact amount. Hence “about 5 mg” means “about 5 mg” and also “5 mg.” Generally, the term “about” includes an amount that would be expected to be within experimental error, such as for example, within 15%, 10%, or 5%.

Section headings are provided solely for the convenience of the reader and do not limit the disclosure.

### Dosage

The results of two in vivo murine studies in Examples 1 and 2, and the results of a first in human phase 1 clinical safety study have provided safe an effective dosage ranges for the denatonium salts of the present disclosure. Based on the conversion formula from mouse to human, the human equivalent dose to 50 mg/kg, BID in mice (the dose used for the treatment in vivo study in Example 2) is 4 mg/kg, BID (or 8 mg/kg/day). Considering the average adult body weight is 60 kg, the corresponding dose is 240 mg, BID (or 480 mg/day). In a first-in-human clinical trial, 240 mg, BID (480 mg/day) was well tolerated. Therefore, for treatment purposes, an upper daily dosage limit (human adult) 600 mg-1000 mg per day is safe and tolerated and lower daily doses have shown efficacy in the pre-clinical murine models provided herein.

Specifically, for treatment of a fatty liver disease, a daily adult human dose of a denatonium salt is from about 10 mg to about 600 mg or from about 0.2 mg/kg to about 10 mg/kg body weight per day. Most preferably, the denatonium salt for an adult is from about 40 mg to about 400 mg or from about 1 mg/kg to about 8 mg/kg body weight per day. The daily dose of the denatonium salt is administered once per day, twice per day, or three times per day. Most preferably, the daily dose of the denatonium salt is administered twice per day.

Specifically, for prevention of fatty liver disease, a daily adult human dose of a denatonium salt is from about 5 mg to about 400 mg or from about 0.1 mg/kg to about 8 mg/kg body weight per day. Most preferably, the denatonium salt for an adult is from about 20 mg to about 200 mg or from about 0.5 mg/kg to about 4 mg/kg body weight per day. The daily dose of the denatonium salt is administered once per day, twice per day, or three times per day. Most preferably, the daily dose of the denatonium salt is administered once per day.

To the extent any material incorporated by reference is inconsistent with the express content of this disclosure, the express content controls.

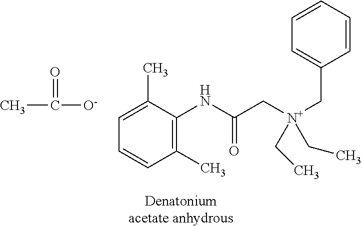

Denatonium acetate anhydrous, or DA is an anhydrous salt such that for every 100 mg of DA, there are 83 mg of denatonium cation, 17 mg of acetate anion.

This Scheme A describes the synthesis of denatonium acetate (DA).

Step 1: Synthesis of Denatonium Hydroxide from Lidocaine

To a reflux apparatus add 25 g of lidocaine, 60 ml of water and 17.5 g of benzyl chloride with stirring and heating in 70-90° C. The solution needs to be heated and stirred in the before given value for 24 h, the solution needs to be cooled down to 30° C. The unreacted reagents are removed with 3×10 mL of toluene. With stirring dissolve 65 g of sodium hydroxide into 65 mL of cold water and add it to the aqueous solution with stirring over the course of 3 h. Filter the mixture, wash with some water and dry in open air. Recrystallize in hot chloroform or hot ethanol.

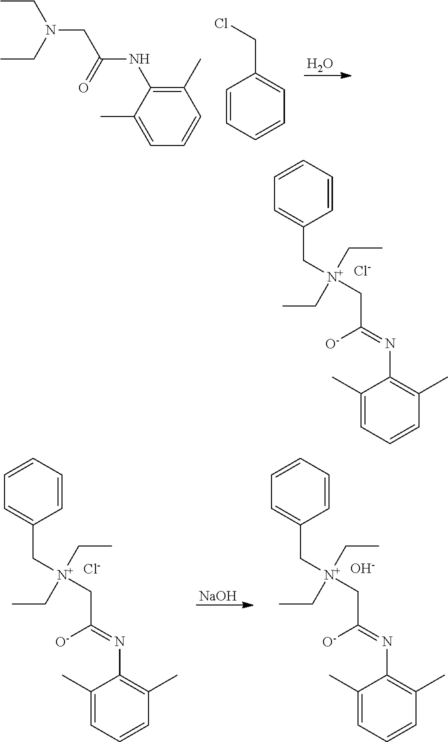

Step 2: Preparation of Denatonium Acetate Anhydrous from Denatonium Hydroxide.

To a reflux apparatus 10 g of denatonium hydroxide (MW: 342.475 g/mol, 0.029 mol), 20 mL of acetone, and 2 g of acetic acid glacial (0.033 mol) dissolved in 15 mL of acetone is added, the mixture is stirred and heated to 35° C. for 3 h. Then evaporated to dryness and recrystallized in hot acetone.

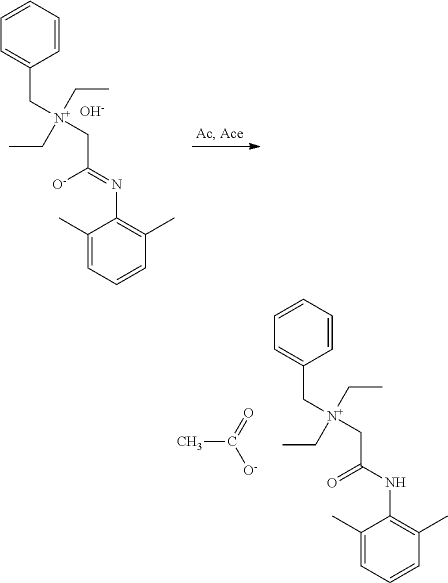

### Formulation of DA Tablet

This provides an immediate release 50 mg granule formulation of denatoniumn acetate (DA) as a free base as an immediate gastric release oral pharmaceutical formulation.

Table 1 shows qualitative and quantitative formulation composition of DA.

| | | | | DA | Limits based on IID | |
|---|---|---|---|---|---|---|
| | | | Quantity | capsule- | Max Potency | |
| | Quality | | (%) | 50 mg | for Unit Dose | |
| Ingredient | Standard | Function | w/w | (mg/cap) | (mg) | Reference |
| Denatonium | In-house | API | 23.55 | 59.03 (20 mg | N/A | N/A |
| acetate | | | | Denatonium base) | | |
| Povidone | USP | Binder | 2.36 | 5.90 | 61.5 | Oral - |
| (KOLLIDON 30) | | | | | | Capsule |
| Sugar Spheres | NF | Substrate | 68.85 | 172.57 | 314.13 | Oral - |
| (VIVAPHARM ® | | | | | | Capsule |
| Sugar Spheres 35-45) | | | | | | |
| Hypromellose | USP | Binder | 3.64 | 9.14 | 150 | Oral - |
| (Methocel | | | | | | Capsule |
| E5 Premium LV | | | | | | |
| Hydroxypropyl | | | | | | |
| Methylcellulose) | | | | | | |
| Talc | USP | Anti- | 1.09 | 2.74 | 14 | Oral - |
| (MicroTalc MP | | tacking | | | | Capsule, |
| 1538 USP Talc) | | agent | | | | coated |
| | | | | | | pellets |
| Talc (extra | USP | Flow aid | 0.50 | 1.25 | 284.38 | Oral - |
| granular) | | | | | | Capsule |
| (MicroTalc MP | | | | | | |
| 1538 USP Talc) | | | | | | |
| Total weight of beads | | | 250.62 | | N/A | N/A |
| Hard Gelatin | USP | Capsule | N/A | 73.3 | 107 | Oral - |
| Capsule Shells; | | shell | | | | Capsule |
| Cap: White | | | | | | |
| Opaque: | | | | | | |
| Body: White | | | | | | |
| Opaque; | | | | | | |
| Size: 1 | | | | | | |

| | | | |
|---|---|---|---|
| Total weight of Filled Capsule | 323.9 | N/A | N/A |

IID, the Inactive Ingredient Database; API, active pharmaceutical ingredient; USP, the US Pharmacopeia; NF, the National Formulary
* Solvents such as Ethyl Alcohol USP 190 Proof (190 Proof Pure Ethyl Alcohol) and purified water (USP) were used for the preparation of drug solution and seal coating dispersion, but are removed during the manufacturing process.

The detailed manufacturing steps are described below.

1. Drug Layering Process—Drug Layered Pellets

Drug layering process was performed in a Fluid bed granulator equipped with the rotor insert (rotor granulator). Drug solution was prepared by solubilizing Povidone K30 (Kollidon 30) and Denatonium Acetate in ethyl alcohol. The drug solution was sprayed tangentially on to the bed of sugar spheres (35/45 mesh) moving in a circular motion in the rotor granulator. The final drug loaded pellets were then dried for ten (10) minutes in the rotor granulator, discharged and screened through a #20 mesh.

2. Seal Coating Process—Seal Coated Pellets

Seal coating dispersion was prepared by separately dissolving Hypromellose E5 in a mixture (1:1) of ethyl alcohol and purified water until a clear solution was obtained. The remaining quantity of ethyl alcohol was then added to the above solution followed by talc. The dispersion was mixed for 20 minutes to allow for uniform dispersion of talc. The seal coating dispersion was sprayed tangentially on to the drug loaded pellets to achieve 5% weight gain. The seal coated pellets were then dried for five (5) minutes in the rotor granulator, discharged and dried further in a tray dryer/oven at 55° C. for 2 hours. The seal coated pellets were then screened through a #20 mesh.

3. Final Blending—Denatonium Immediate Release (IR) Pellets

The seal coated pellets were blended with talc screened through mesh #60 using a V-Blender for ten (10) minutes and discharged. The blended seal coated beads, Denatonium IR Pellets, were used for encapsulation.

4. Encapsulation—Denatonium Capsules, 50 mg

The Denatonium IR pellets, 50 mg, were filled into size 1, white opaque hard gelatin capsules using an auto capsule filling machine. Capsules were then passed through an in-line capsule polisher and metal detector. In-process controls for capsule weight and appearance was performed during the encapsulation process. Acceptable quality limit (AQL) sampling and testing was performed by Quality Assurance (QA) on a composite sample during the encapsulation process. Finished product composite sample was collected and analyzed as per specification for release testing.

5. Packaging—Capsules, 50 mg—30 Counts

The 50 mg capsules were packaged in 30 counts into 50/60 cc White HDPE round 5-line bottles with 33 mm White CRC Caps. The bottles were torqued and sealed using an induction sealer.

TABLE 2
Summary Comparison of NASH data from multiple studies:

| | | | | NASH animal | Dose | | Findings of |
|---|---|---|---|---|---|---|---|
| | | | | model(s) | schedule and | Findings of | metabolic |
| | | | | used in pre- | treatment | histopathological | biomarker |
| Drug Name | Developer | Modality | MOA | clinical study | period | examination | measurements |
| DA or | Aardvark | Small | Bitter taste | Male | 30 mg/kg, | Significantly | Lowered serum |
| ARD-101 | Therapeutics | molecule | receptor | C57BL/6 | PO, QD | decreased liver | levels of fasting |
| | | | agonist | mice fed | from | weight of | glucose (−9%), |
| | | | | AMLN diet | beginning | animals | HbA1c (−6.7%), |
| | | | | (40 kcal % | of the study | (by 10%) | ALT (−43%), |
| | | | | fat, 20 | for 48 | Significantly | AST (−9.5%), |
| | | | | kcal % | weeks | improved | and bile |
| | | | | fructose, | | steatosis and | acid (−16.2%) |
| | | | | and 2% | | fibrosis on | Increased |
| | | | | cholesterol) | | histopathology | serum levels |
| | | | | for 48 weeks | | | of GLP-1 and |
| | | | | | | | GLP-2 |
| | | | | | | | Lowered serum |
| | | | | | | | levels of |
| | | | | | | | inflammatory |
| | | | | | | | markers |
| MET409 | Metacrine | Small | Farnesoid | Male | 10 mg/kg, | Significantly | Lowered |
| | | molecule | X receptor | C57BL/6 | PO, QD | improved | plasma C4, |
| | | | (FXR) | mice fed | after 34- | steatosis with | liver TG, ALT, |
| | | | agonist | AMLN diet | week | all treatment | and AST |
| | | | | for 34 weeks | NASH | courses and | |
| | | | | | induction | inflammation | |
| | | | | | for 2, 4, | with 4- or 8- | |
| | | | | | or 8 weeks | week treatment | |
| | | | | | | Significant | |
| | | | | | | improvement | |
| | | | | | | on fibrosis | |
| | | | | | | only observed | |
| | | | | | | with 8-week | |
| | | | | | | treatment | |
| Cenicriviroc | Takeda and | Small | CCR2/CCR5 | Male | 10 | Significantly | Inhibited |
| | Tobira | molecule | Inhibitor | C57BL/6N | mg/kg/day | improved | intrahepatic |
| | Therapeutics | | | mice fed | and 30 | fibrosis on | accumulation |
| | | | | choline- | mg/kg/day | histopathology | of inflammatory |
| | | | | deficient, L- | for 4 weeks | only with 30 | macrophages |
| | | | | amino acid- | and 20 | mg/kg/day for | with 30 |
| | | | | defined, | mg/kg/day | 14 weeks | mg/kg/day for |
| | | | | high-fat diet | and 30 | | 4 or 14 weeks, |
| | | | | (CDAHFD)f | mg/kg/day | | and increased |
| | | | | or 4 or 14 | for 14 | | the frequency |
| | | | | weeks | weeks, IP | | of intrahepatic |
| | | | | | | | anti-inflammatory |
| | | | | | | | macrophages |
| | | | | | | | with 30 |
| | | | | | | | mg/kg/day for |
| | | | | | | | 14 weeks |
| Elafibranor | Genfit | Small | PPARα | Male | 30 mg/kg, | Significantly | No effects on |
| (GFT505) | | molecule | and | C57BL/6J | PO, QD for | reduced | liver TG, total |
| | | | PPARδagonist | mice fed | 8 weeks | histopathological | cholesterol |
| | | | | with the | | scores of | contents, or |
| | | | | AMLN diet | | hepatic | plasma levels |
| | | | | for 50 weeks | | steatosis and | of ALT, AST, |
| | | | | | | inflammation, | TG or total |
| | | | | | | as well as | cholesterol. |
| | | | | | | fibrosis | |
| | | | | | | severity. | |
| The | Genfit | Small | PPARα | Male | Elafibranor | The | A strong |
| combination | | molecule + | and | C57BL/6J | (10 mg/kg/ | combination | decrease in |
| of | | Recombinant | PPARδagonist + | mice fed | day, PO) + | decreased the | liver |
| elafibranor | | protein | GLP-1 | with the | Semaglutide | NAS score by | triglycerides |
| and | | | receptor | AMLN diet | (0.3 nmol/kg, | 3 stages in | (−56%), in the |
| semaglutide | | | agonist | for 35 weeks | SC) for 12 | 14% of mice, | number of |
| | | | | | weeks | and by 2 | inflammatory |
| | | | | | | stages in 44% | foci (−59%) and |
| | | | | | | of mice. | in plasma ALT |
| | | | | | | | (−60%), was |
| | | | | | | | also observed in |
| | | | | | | | the combination |
| | | | | | | | group. |
| | | | | | | | Transcriptomic |
| | | | | | | | analysis |
| | | | | | | | revealed that |
| | | | | | | | both drugs |
| | | | | | | | synergized to |
| | | | | | | | specifically |
| | | | | | | | reduce the |
| | | | | | | | inflammatory |
| | | | | | | | infiltration in |
| | | | | | | | the liver |
| ALT- | Altimmune | Recombinant | Dual | Male | 5 or 10 | Significantly | Significantly |
| 801 | | protein | GLP-1/ | C57BL/6J | nmol/kg, | decreased liver | decreased liver |
| | | | glucagon | mice fed | SC for 12 | weight and | TG and total |
| | | | receptor | with the | weeks | NAS overall | cholesterol |
| | | | agonist | AMLN diet | | score at both | levels, plasma |
| | | | | for 32 weeks | | doses | ALT level, and |
| | | | | | | | liver content of |
| | | | | | | | fibrosis markers. |
| Semaglutide | Novo | Recombinant | GLP-1 | Male | 10 nmol/kg, | Significantly | Significantly |
| | Nordisk | protein | receptor | C57BL/6J | SC for 12 | decreased liver | decreased liver |
| | | | agonist | mice fed | weeks | weight and | TG and total |
| | | | | with the | | NAS overall | cholesterol |
| | | | | AMLN diet | | score at 10 | levels, plasma |
| | | | | for 32 weeks | | nmol/kg | ALT level, and |
| | | | | | | | liver content of |
| | | | | | | | fibrosis markers. |
| Obeticholic | Intercept | Small | FXR | Male | 30 mg/kg, | Significantly | Significantly |
| acid | Pharmaceuticals | molecule | agonist | C57BL/6J | PO, QD for | reduced | reduced total |
| (INT-747) | Inc. | | | mice fed | 8 weeks | histopathological | liver TG and |
| | | | | with the | | scores of hepatic | total |
| | | | | AMLN diet | | steatosis and | cholesterol, |
| | | | | for 50 weeks | | inflammation. | collagen 1a1, |
| | | | | | | Significantly | and galectin-3 |
| | | | | | | reduced liver | content. |
| | | | | | | weight. | Significantly |
| | | | | | | | reduced plasma |
| | | | | | | | total cholesterol |
| Selonsertib | Gilead | Small | Apoptosis | Male | Treatment | Significantly | Reduced serum |
| (GS-4997) | Sciences | molecule | signal- | C57BL/6 | started after | reduced liver | and liver |
| | | | regulating | mice fed a | FF diet | steatosis, | cholesterol by |
| | | | kinase 1 | fast food | induction | fibrosis, and | 14% and 45%, |
| | | | (ASK1) | diet (FF | for 240 | insulin | respectively, |
| | | | inhibitor | diet) high | days. Dose | resistance | and strongly |
| | | | | in fat. | unknown | | reduced serum |
| | | | | cholesterol, | | | bile acids |
| | | | | and sugar | | | including |
| | | | | for 330 days | | | cholate (91% |
| | | | | | | | reduction) and |
| | | | | | | | deoxycholate |
| | | | | | | | (90% reduction). |
| Aramchol | Galmed | Small | Stearoyl- | C57BL/6 | 5 mg/kg/day | Reduced | No effects on |
| | Pharmaceuticals | molecule | CoA | male mice | PO for the | features of | ALT, AST, or |
| | | | desaturase-1 | fed with the | last 2 weeks | steatohepatitis | TG. But |
| | | | (SCD-1) | methionine- | | and fibrosis | decreased protein |
| | | | inhibitor | and choline- | | | expression of |
| | | | | deficient | | | COL1A1 in the |
| | | | | (MCD) diet | | | liver |
| | | | | for 4 weeks | | | |
| Pegbelfermin | Bristol-Myers | Recombinant | PEGylated | STAM | 3 mg/kg, | Significantly | Significantly |
| (BMS-986036) | Squibb | protein | human | model: | twice | decreased | improved |
| | | | fibroblast | Streptozotocin- | weekly, SC | mean grades | whole blood |
| | | | growth | injected | for 2 weeks | for steatosis, | glucose (−30%), |
| | | | factor 21 | 2-day old | (start at | lobular | body weight |
| | | | (FGF21) | male | Week 7) | inflammation, | (−7.9%), and |
| | | | analogue | C57BL/6 | | and | liver/body |
| | | | | mice fed | | hepatocellular | weight ratio |
| | | | | high-fat diet | | ballooning | (−20%), as well |
| | | | | for over 7 | | | as liver and |
| | | | | weeks | | | plasma |
| | | | | | | | triglycerides. |
| | | | | | | | Also |
| | | | | | | | significantly |
| | | | | | | | decreased the |
| | | | | | | | mean serum |
| | | | | | | | fibrosis |
| | | | | | | | biomarker Pro- |
| | | | | | | | C3 by 44% |
| Emricasan | Conatus | Small | Pancaspase | Male | 0.3 mg/kg/ | Improved | Decreased |
| | Pharmaceuticals/ | molecule | inhibitor | C57BL/6J | day PO for | inflammation, | serum levels of |
| | Novartis | | | mice fed | 20 weeks | ballooning, | glucose, |
| | | | | high fat diet | | and fibrosis on | HOMA-IR, |
| | | | | for 20 weeks | | histopathology | cholesterol, |
| | | | | | | | ALT, and AST. |
| | | | | | | | Decreased |
| | | | | | | | fibrotic and |
| | | | | | | | inflammatory |
| | | | | | | | gene signature |
| | | | | | | | in mice |
| EDP-305 | Enanta | Small | FXR | Male | 10 mg/kg or | High-dose (30 | High dose |
| | Pharmaceuticals | molecule | agonist | C57BL/6 | 30 mg/kg | mg/kg) halted | significantly |
| | | | | mice fed a | daily PO | fibrosis | decreased |
| | | | | CDAHFD | starting | progression on | fibrogenic gene |
| | | | | diet for 12 | from the | histopathology | expression in |
| | | | | weeks | beginning | in CDAHFD | CDAHFD mice |
| | | | | | of week 6 | mice | |
| Tropifexor | Novartis | Small | FXR | STAM | STAM | STAM model: | AMLN model: |
| | | molecule | agonist | model: | model: | tropifexor | tropifexor |
| | | | | Streptozotocin- | 0.03-0.3 | treatment | showed dose- |
| | | | | injected | mg/kg from | showed | dependent |
| | | | | 2-day old | Week 9 | significant | reduction in |
| | | | | C57BL/6J | AMLN | decrease in | ALT/AST |
| | | | | mice fed | model: | NAS at | levels relative |
| | | | | high-fat diet | 0.03-0.9 | doses ≥0.1 | to controls and |
| | | | | for weeks 4-12 | mg/kg for | mg/kg AMLN | dramatically |
| | | | | AMLN | the last 4 | model: | reduced mRNA |
| | | | | model: | weeks | tropifexor | levels of |
| | | | | C57BL/6J | | reduced | fibrogenic |
| | | | | mice placed | | inflammation, | markers and |
| | | | | on AMLN | | steatosis, and | hepatic |
| | | | | diet for 30 | | fibrosis in | inflammation |
| | | | | weeks | | AMLN mice. | cell populations |
| | | | | | | Steatosis and | |
| | | | | | | inflammation | |
| | | | | | | were | |
| | | | | | | completely | |
| | | | | | | resolved at | |
| | | | | | | doses ≥0.3 | |
| | | | | | | mg/kg. | |
| Saroglitazar | Zydus-Cadila | Small | PPARα | Male | 3 mg/kg PO | Saroglitazar | Saroglitazar at |
| | | molecule | and PPARγ | C57BL/6 | for 12 weeks | (3 mg/kg) | 3 mg/kg |
| | | | agonist | mice fed a | (starting | induced | reduced serum |
| | | | | CDAHFD | from 8 | reversal of | levels of liver |
| | | | | diet for 20 | weeks after | hepatic | damage and |
| | | | | weeks | initiation of | steatosis, | inflammation |
| | | | | | the CDAHFD | reduced or no | markers, |
| | | | | | diet) | vacuolation | including ALT |
| | | | | | | and ballooning | (60%), AST |
| | | | | | | and there was | (43%), and |
| | | | | | | significant | MCP1 (45%). |
| | | | | | | reduction in | Liver lipid (TG) |
| | | | | | | the severity of | accumulation |
| | | | | | | inflammation | and collagen |
| | | | | | | | content were |
| | | | | | | | also |
| | | | | | | | significantly |
| | | | | | | | (79% and 41% |
| | | | | | | | respectively) |
| | | | | | | | attenuated by |
| | | | | | | | saroglitazar |
| | | | | | | | treatment. |
| | | | | | | | Saroglitazar |
| | | | | | | | also reduced |
| | | | | | | | liver TNFα |
| | | | | | | | levels and |
| | | | | | | | hepatic |
| | | | | | | | fibrogenic and |
| | | | | | | | inflammatory |
| | | | | | | | gene |
| | | | | | | | expression in |
| | | | | | | | CDAHFD mice |
| Belapectin | Galectin | Polysaccharide | Galectin 3 | STAM | 60 mg/kg | Decreased fat | Treatment with |
| (GR-MD-02) | Therapeutics | polymer | inhibitor | model: | IV twice a | deposition, | GR-MD-02 |
| | | | | Streptozotocin- | week for 4 | hepatocellular | reduced the |
| | | | | injected | weeks | ballooning, and | expression of |
| | | | | 2-day old | starting | inflammatory | pathological |
| | | | | male | from Week | infiltrate, and | indicators |
| | | | | C57BL/6 | 6 (early | significantly | including |
| | | | | mice fed | treatment | improved NAS | iNOS, CD36, |
| | | | | high-fat diet | cohort) or | score in the | and a-smooth |
| | | | | for weeks | Week 9 | early treatment | muscle actin |
| | | | | 10-13 weeks | (late | cohort. The | |
| | | | | | treatment | late treatment | |
| | | | | | cohort). | cohort GR- | |
| | | | | | | MD-02 group | |
| | | | | | | showed | |
| | | | | | | improvement | |
| | | | | | | in the NAS | |
| | | | | | | versus vehicle | |
| | | | | | | control, | |
| | | | | | | although the | |
| | | | | | | value did not | |
| | | | | | | reach | |
| | | | | | | significance. | |
| | | | | | | Treatment | |
| | | | | | | with GR-MD- | |
| | | | | | | 02 markedly | |
| | | | | | | reduced the | |
| | | | | | | deposition of | |
| | | | | | | collagen in | |
| | | | | | | both the early | |
| | | | | | | and late | |
| | | | | | | treatment | |
| | | | | | | cohorts. | |
| Firsocostat | Gilead | Small | Acetyl- | Male | GS-9674: | The | The |
| (GS-0976) + | Sciences | molecule | CoA | C57BL/6 | 10 mg/kg, | combination | combination |
| Cilofexor | | | carboxylase | mice fed a | QD, PO | significantly | significantly |
| (GS-9674) | | | (ACC) | fast-food | GS-0976: | reduced | reduced liver |
| | | | inhibitor + | diet (FFD) | 0.5 mg/kg, | steatosis on | TG and |
| | | | Farnesoid X- | enriched in fat, | BID, PO (a | histopathology. | cholesterol |
| | | | activated | cholesterol, | structural | | levels. |
| | | | receptor | and sugar | analog was | | The |
| | | | agonist | for 6 months | used in the | | combination |
| | | | | | study | | significantly |
| | | | | | Treatment | | reduced hepatic |
| | | | | | started from | | gene |
| | | | | | Month 5 and | | expression of |
| | | | | | continued | | fibrosis and |
| | | | | | for 28 days | | liver injury |
| | | | | | | | markers, and |
| | | | | | | | plasma level of |
| | | | | | | | bile acids. |
| Lanifibranor | Inventiva | Small | Pan-PPAR | C57Bl6/J | 10 or 30 | IVA337 | IVA337 also |
| (IVA337) | Pharma | molecule | (peroxisome | mice fed a | mg/kg, PO, | prevented | significantly |
| | | | proliferator- | MCD diet | QD for 3 | steatosis and | reduced plasma |
| | | | activated | for 3 weeks | weeks | inflammation on | alanine |
| | | | receptors) | | | histopathology. | aminotransferase |
| | | | agonist | | | | levels, |
| | | | | | | | decreased |
| | | | | | | | serum as well |
| | | | | | | | as liver |
| | | | | | | | triglyceride |
| | | | | | | | levels, and |
| | | | | | | | inhibited the |
| | | | | | | | induction of |
| | | | | | | | profibrotic and |
| | | | | | | | fibrotic genes. |
| Resmetirom | Madrigal | Small | Thyroid | C57BL/6 | 0.3-3 | No detectable | Improved |
| (MGL-3196) | Pharmaceuticals | molecule | hormone | mice treated | mg/kg/day | steatosis, | insulin |
| | | | receptor | with 60% | PO for 24 | fibrosis or | sensitivity, and |
| | | | beta | high fat diet | days | inflammation | reduced serum |
| | | | agonist | for 36 weeks | | in | levels of ALT |
| | | | | | | MGL-3196 | (46%), free |
| | | | | | | treated livers | fatty acids |
| | | | | | | | (30%), and |
| | | | | | | | cholesterol |
| | | | | | | | (67%). |
| Azemiglitazone | Cirius | Small | Mitochondrial | Male | 30 mg/kg/day | Treatment | MSDC-0602K |
| (MSDC0602K) | Therapeutics | molecule | membrane | DIAMOND ™ | PO QD for | with MSDC- | dramatically |
| | | | transport | mice fed | the last 8 | 0602K | reduced serum |
| | | | protein | high fat | weeks (16- | significantly | levels of fasting |
| | | | modulator | sugar water | 24 weeks) | decreased | insulin, and |
| | | | | diet | | ballooning; the | AST and ALT. |
| | | | | (WDSW) | | steatosis- | |
| | | | | for 24 weeks | | activity- | |
| | | | | | | fibrosis (SAF) | |
| | | | | | | score was | |
| | | | | | | significantly | |
| | | | | | | lower in the | |
| | | | | | | MSDC- | |
| | | | | | | 0602K-treated | |
| | | | | | | group and was | |
| | | | | | | reduced to the | |
| | | | | | | level of | |
| | | | | | | baseline | |
| | | | | | | controls. The | |
| | | | | | | NAFLD | |
| | | | | | | Activity Score | |
| | | | | | | (NAS) trended | |
| | | | | | | lower in the | |
| | | | | | | MSDC- | |
| | | | | | | 0602K-treated | |
| | | | | | | group. The | |
| | | | | | | NASH CRN | |
| | | | | | | fibrosis score | |
| | | | | | | in the MSDC- | |
| | | | | | | 0602K-treated | |
| | | | | | | group was 0. | |
| | | | | | | Significantly | |
| | | | | | | fewer MSDC- | |
| | | | | | | 0602K-treated | |
| | | | | | | mice | |
| | | | | | | progressed | |
| | | | | | | from simple | |
| | | | | | | steatosis to | |
| | | | | | | NASH | |
| | | | | | | compared to | |
| | | | | | | the control | |
| | | | | | | group. | |
| Aldafermin | NGM | Recombinant | FGF19 | FXR- | NGM282 | NGM282 | NGM282 |
| (NGM282) | Biopharmaceuticals | protein | analogue | deficient | delivered by | reduced liver | reduced serum |
| | | | | mice placed | adeno- | weight and | levels of ALT |
| | | | | on a high- | associated | spleen weight | and AST, and |
| | | | | fat, high- | viral vector | in HFFCD- | decreased |
| | | | | fructose, | (AAV) at | fed, FXR- | hepatic |
| | | | | highcholesterol | Week 16 | deficient mice | expression of |
| | | | | diet | | NGM282 did | inflammatory |
| | | | | (HFFCD) | | not impact | and fibrotic |
| | | | | for 50 weeks | | steatosis in | biomarkers |
| | | | | | | HFFCD-fed, | |
| | | | | | | FXR-deficient | |
| | | | | | | mice, but | |
| | | | | | | reduced | |
| | | | | | | hepatocellular | |
| | | | | | | fibrosis | |
| VK2809 | Viking | Small | Thyroid | A mouse | 10 | VK2809 | VK2809 |
| | Therapeutics | molecule | hormone | model of | mg/kg/day | significantly | significantly |
| | | | receptor | diet-induced | PO for eight | improved NAS | improved liver |
| | | | beta | NASH | weeks | score (a 40% | contents of |
| | | | agonist | | | mean | triglyceride, |
| | | | | | | improvement) | cholesterol, |
| | | | | | | and liver | collagen, and |
| | | | | | | fibrosis on | hydroxyproline. |
| | | | | | | histopathology. | It also |
| | | | | | | Improvements | significantly |
| | | | | | | in all | reduced plasma |
| | | | | | | subcomponents | triglycerides |
| | | | | | | of NAS | and cholesterol. |
| | | | | | | (inflammation, | |
| | | | | | | ballooning, | |
| | | | | | | and steatosis) | |
| | | | | | | were observed | |
| | | | | | | in VK2809- | |
| | | | | | | treated animals | |

Table 2 shows a wide range of different results in widely different NASH in vivo models. This makes it difficult to do direct comparisons of the data. The study corresponding to the first row (called Aardvark Therapeutics) is provided in Example 1 below and the figures provided with this disclosure. It should be noted that unlike many prior art NASH models, the present disclosure did a much longer daily dosing of 48 weeks as it is estimated that at least 30 weeks of AMLN diet of time is needed to induce full fibrosis NASH in a mouse model. Many of the other studies were shorter in duration. Accordingly, the Example 1 study is more predictive of an effect for a method of prevention or slowing of progression of NASH and similar liver diseases characterized by liver fibrosis.

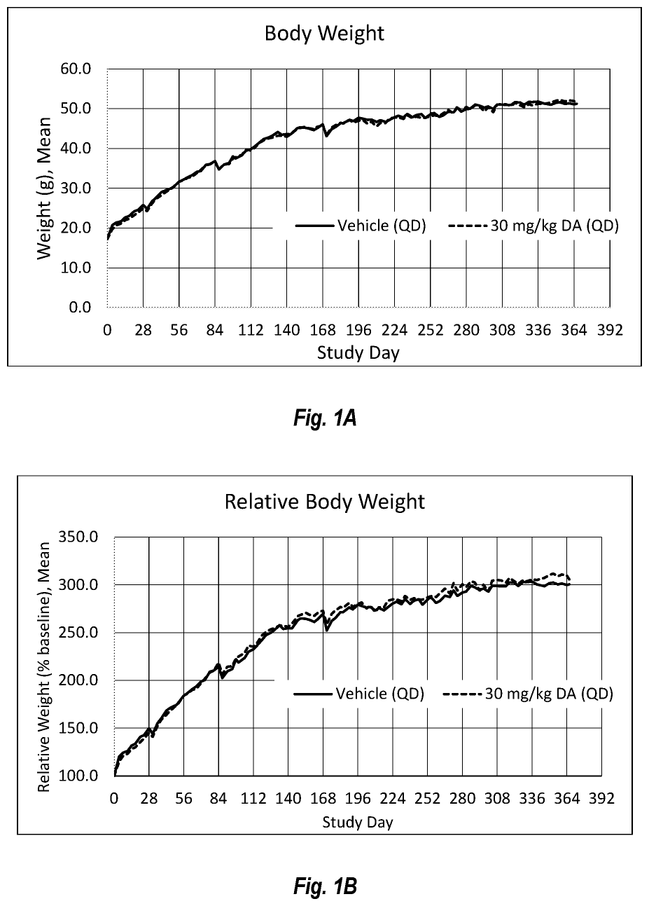

The studies that emphasized weight loss as a model for NASH treatment, seem to be more directed toward treating existing NASH conditions. Therefore, the present disclosure further incorporates by reference U.S. patent application Ser. No. 17/132,580 filed 23 Dec. 2020. In particular, Example 9 and the accompanying figures present the results of a weight loss study with a higher dose of DA (23.1 mg/kg BID corresponding to 46.2 15 mg/kg/day DA or 37.4 mg/kg/day dry denatonium) showing a significant reduction of weight gain. In contrast to the DIO shorter duration weight loss study with approximately double the daily dose of DA, the present NASH prevention study with a lower dose of DA showed no difference in weight gain from vehicle control over 48 weeks (FIG. 1 herein). Therefore, this comparison means either a higher dose per day of DA is needed for treatment versus prevention or greater frequency of dosing is needed for treatment or both aspects are needed for treatment. But the study in Example 1 clearly shows that a similar dose but only once per day (not twice) is sufficient for prevention and slowing progression of NASH but not for weight loss that may be more of a marker for treatment of NASH. Therefore, the dosage range of denatonium salt for a method of treatment of NASH and related liver diseases is from about 1.0 mg/kg/day to about 12 mg/kg/day; preferably from about 2 mg/kg/day to about 8 mg/kg/day; and most preferably from about 3 mg/kg/day to about 6 mg/kg/day. Therefore, the dosage range of denatonium salt for a method of prevention and a method of slowing progression of NASH and related liver diseases is from about 0.1 mg/kg/day to about 6 mg/kg/day, preferably from about 0.25 mg/kg/day to about 4 mg/kg/day; and most preferably from about 0.5 mg/kg/day to about 3 mg/kg/day.

### EMBODIMENTS

In a first aspect, the present disclosure provides a method (Method 1) for preventing or preventing progression of a fatty liver disease (e.g., nonalcoholic steatohepatitis (NASH), alcoholic steatohepatitis (ASH), non-alcoholic fatty liver disease (NAFLD), and HIV-associated steatohepatitis) with or without liver fibrosis, comprising administering an effective amount of a pharmaceutical composition comprising a comprising a denatonium salt, wherein the denatonium salt is selected from the group consisting of denatonium acetate (DA), denatonium citrate, denatonium maleate, denatonium saccharide, and denatonium tartrate and wherein the daily dose of the denatonium salt for preventing or preventing progression of a fatty liver disease is from about 0.1 mg/kg/day to about 8 mg/kg/day QD, BID or TID. Preferably, the pharmaceutical composition further comprises from about 0.5 g to about 5 g acetic acid. More preferably, the dosage per day of the acetic acid for an adult is from about 1.5 g to about 3 g.

The present disclosure further provides the following embodiments of the method for preventing or preventing progression of a fatty liver disease:
1.1 Method 1, wherein the fatty liver disease is NASH.
1.2 Method 1 or 1.1, wherein the fatty liver disease is NASH or NAFLD or ASH.
1.3 Any of the preceding Methods, wherein the denatonium salt is denatonium acetate.
1.4 Any of the preceding Methods, wherein the denatonium salt is denatonium citrate.
1.5 Any of the preceding Methods, wherein the denatonium salt is denatonium maleate.
1.6 Any of the preceding Methods, wherein the daily dose of the denatonium salt is 0.2 mg/kg/day QD.
1.7 Any of the preceding Methods, wherein the daily dose of the denatonium salt is 1.0 mg/kg/day QD.
1.8 Any of the preceding Methods, wherein the daily dose of the denatonium salt is 4.0 mg/kg/day QD.

In a second aspect, the present disclosure provides a method (Method 2) for treating a fatty liver disease (e.g., nonalcoholic steatohepatitis (NASH), alcoholic steatohepatitis (ASH), non-alcoholic fatty liver disease (NAFLD), or HIV-associated steatohepatitis) with or without liver fibrosis, comprising administering an effective amount of a pharmaceutical composition comprising a denatonium salt, wherein the denatonium salt is selected from the group consisting of denatonium acetate (DA), denatonium citrate, denatonium maleate, denatonium saccharide, and denatonium tartrate, and wherein the daily dose of the denatonium salt for treating an existing fatty liver disease is from about 1.0 mg/kg/day to about 10 mg/kg/day administered QD, BID or TID. Preferably, the pharmaceutical composition further comprises from about 0.5 g to about 5 g acetic acid. More preferably, the dosage per day of the acetic acid for an adult is from about 1.5 g to about 3 g.

The present disclosure further provides the following embodiments of the method for treating a fatty liver disease:
2.1 Method 2, wherein the fatty liver disease is NASH.
2.2 Method 2 or 2.1, wherein the fatty liver disease is NASH or NAFLD or ASH.
2.3 Any of the preceding Methods, wherein the denatonium salt is denatonium acetate.
2.4 Any of the preceding Methods, wherein the denatonium salt is denatonium citrate.
2.5 Any of the preceding Methods, wherein the denatonium salt is denatonium maleate.
2.6 Any of the preceding Methods, wherein the daily dose of the denatonium salt is 2.5 mg/kg/day BID.
2.7 Any of the preceding Methods, wherein the daily dose of the denatonium salt is 5 mg/kg/day BID.
2.8 Any of the preceding Methods, wherein the daily dose of the denatonium salt is 7.5 mg/kg/day BID.

### FDA Guidance for Clinical Trials for Treating NASH

On 29 Jan. 2021, the Food and Drug Administration (FDA) gave a short seminar on NASH with fibrosis how treatment drug candidates can show efficacy in animal models and clinical trials. The FDA confirmed that NASH (with fibrosis, hereinafter, NASH) is a serious condition and that a clinical use of surrogate endpoints can predict clinical benefit. Although in animal studies (such as provided in Example 1, herein) histopathological examination is a better proof of treatment, prevention and progression of disease benefit (depending on the length of the animal study). Therefore, in clinical trials, the FDA will accept surrogate endpoints and liver biopsy as means for showing clinical benefit (or lack thereof). The FDA recognized that NASH drug development challenges are due to a gradual and slow progression of chronic inflammatory changes in the liver, and that any NASH drug for prevention of full NASH (advanced liver fibrosis) or treatment or slowing progression are potential lifelong treatments. As for a surrogate endpoint, the FDA has suggested histopathology as “reasonably likely to predict clinical benefit.” The FDA indicated that NASH advanced liver “fibrosis stage, but no other histologic feature of steatohepatitis, has been associated independently with increased mortality, transplantation, and liver-related events.” (citing Angulo et al. Gastroenterology, 149:389-397, 2015).

In conducting clinical trials, the FDA suggests that early-stage trials use noninvasive disease-specific biomarkers (e.g., an aminotransferase), total bilirubin, and radiographic modalities (such as elastography, MRI-PDFF) to assess liver stiffness. For approvals, the FDA will accept improvement in liver histology. “Liver biopsy is a surrogate based on research demonstrating that improvement in histology is likely predictive of an improved clinical outcome in NASH patients.” Liver fibrosis is graded as stage 0 (none), stage, stage 2, stage 3 and stage 4 (cirrhosis). The NASH recommended endpoints are (1) resolution of steatohepatitis AND no worsening of liver fibrosis; OR (2) improvement in liver fibrosis AND no worsening of steatohepatitis; OR (3) both resolution of steatohepatitis and improvement in fibrosis.

### Example 1

This example provides the results of a 52 week study of lower dose DA (30 mg/kg per day QD by gavage) in mice where NASH conditions were induced by diet. The data for an animal-induced NASH of such a long duration (48-52 weeks) can be interpreted to support methods for preventing fatty liver diseases, including NASH, and methods of slowing progression of fatty liver diseases, including NASH. Male C57BL/6 mice (4 weeks old at study start) were maintained on a high-fat “Western diet” (AMLN diet) (DIO-NASH) (D09100301, Research Diet, USA) (40% fat (18% trans-fat), 40% carbohydrates (20% fructose) and 2% cholesterol, 40 kcal % fat, 20 kcal % fructose, and 2% cholesterol) following arrival, inducing non-alcoholic steatohepatitis (NASH). Following acclimation, Groups 1 and 2 (n=22 each) were dosed by intragastric oral gavage (PO) with (respectively) vehicle (distilled water) or denatonium acetate (DA) at 30 mg/kg. Doses were administered once daily (QD) for a full year (through Day 366) at dose volumes of 1 mL/kg. Body weights and clinical observations were monitored throughout the study. Blood was collected from fasted animals at baseline (Day −1) and regularly during the study (Weeks 4, 12, 24, and 48), processed to serum or plasma, and evaluated for metabolic parameters [glucose, insulin, hemoglobin A1c (HbA1c), and glucagon-like peptides 1 and 2 (GLP-1 and -2)], selected serum chemistry parameters [Ala aminotransferase (ALT), Asp aminotransferase (AST), albumin (ALB), and total bile acids (BA)], and multiple cytokines (32 total). After 52 weeks of QD dosing, fasted animals were bled and euthanized, and livers were collected and weighed. Terminal serum samples were assessed for low- and high-density cholesterol (LDL and HDL, respectively) and triglycerides (TGA); liver was assessed for total cholesterol (TC), TGA, and free fatty acids (FFA).

Three animals were found dead during the study, including one vehicle-dosed mouse (on Week 51) and two DA-dosed mice (one each on Weeks 12 and 49). None of these three animals exhibited clinical signs prior to being found dead, although one of the DA-dosed mortalities had shown decreasing body weight from Week 44 and the other DA-dosed mortality was notably heavier than the other animals from the study start. It was unclear if either of the Group-2 (DA-dosed) deaths were test article related. No other clinical observations were noted during the study interval.

For both groups, body weights exhibited a mean increase of approximately 200% (i.e., tripling in weight) from baseline during the year-long study. Notably, body weights (whether expressed as absolute or relative values) did not differ in a statistically significant manner between the two groups during the study, with the sole exception of relative body weights at the first time point (Day 4) after the start of dosing (when relative body weights were lower in the DA-dosed mice of Group 2).

For in-life blood samples, mice dosed with DA showed (compared to vehicle) significant differences as follows:
Metabolic parameters: attenuation of fasting glucose and GLP-1 level at Week 24, and of HbA1c at Weeks 12 and 48; and elevation of insulin levels at Weeks 4 and 12. Significant differences were not seen in GLP-2 levels (assessed only at Week 48).
Serum chemistry: attenuation of ALT at Weeks 24 and 48, and of AST and BA at Week 24. Significant differences were not seen in serum ALB levels.
Cytokines: attenuation of IP-10 and MIP-1B at Week 4; IL-13 at Week 12; MIP-1a at Week 24; IL-6, IL-9, and KC at Week 48; and MIG at Weeks 4 and 48. Significant treatment-related differences were not seen in the levels of any of the other (n=24) assessed cytokines.

At necropsy after one year of QD dosing, relative (body weight-normalized) but not absolute liver weight was attenuated in the DA-treated mice (compared to vehicle-dosed animals).

Terminal blood samples revealed that serum TGA levels, were significantly attenuated in the DA-treated mice (compared to vehicle-dosed animals) after one year of daily dosing. In the necropsy liver samples, TGA levels, but not those of TC or FFA, were significantly attenuated in the DA-treated mice (compared to vehicle-dosed animals).

Thus, one year of DA dosing (30 mg/kg, PO, QD) of AMLN diet-induced NASH mice did not appear to have adverse effects on survival, body weight, or clinical signs. Compared to vehicle, DA resulted in significant changes in a number of metabolic parameters (fasting glucose, GLP-1, HbA1c, and insulin), serum chemistry (ALT, AST, and BA), and a subset of cytokines (IL-6, IL-9, IL-13, IP-10, KC, MIG, MIP-1α, and MIP-1B) at selected in-life time points. After one year of QD dosing, DA-dosed mice exhibited (compared to vehicle) attenuation of relative liver weight and of TGA levels in both serum and liver. Male C57BL/6 mice (n=44) were purchased from Taconic Biosciences, Inc. (Rensselaer, NY), as 4-week-old animals. Following arrival, animals were weighed using an electronic balance (Ohaus SCOUT® PRO, Parsippany, NJ), given a clinical examination to ensure that the mice were in good condition, and group-housed (up to 4 per cage). The animals were maintained in HEPA-filtered static cages using SaniChip bedding 7090A (Harlan Teklad, Hayward, CA). Animal room controls were set to maintain temperature and relative humidity at 22° C.±4° C. and 50% 20%, respectively. Housing rooms were on a 12:12 light/dark cycle. Animals were acclimated on site for at least 3 days prior to entry onto the study. Following arrival and throughout the study, mice were provided with ad libitum access to water (via water bottles) and (except as noted for fasting) to a high-fat “Western diet” (AMLN diet, No. D09100301; Research Diets, New Brunswick, NJ) containing 40 kcal % fat, 20 kcal % fructose, and 2% cholesterol.

DA was formulated at 30 mg/mL in DW. Solid DA was weighed and added to the appropriate volume of DW; the solution was mixed well and inspected visually to ensure that there was no precipitate and to verify that the test article was completely solubilized. The DA dosing solution was prepared freshly each week and stored refrigerated at 4° C. between use in daily dose administrations.
On Day −1 (i.e., one day before the start of dosing); and on Weeks 4, 12, 24, and 48 (i.e., after one, 3, 6, and 12 months of dosing), blood and fecal samples were collected from fasted animals.
Fecal samples (4 pellets per cage of 3-4 mice) were collected directly collected from the floor of each cage, divided into two aliquots, and stored at −80° C. for future microbiota and microbiome analysis. Results of those analyses are not included in this report.
Blood (100 μL/animal) was collected from each animal via the submandibular route. All blood samples were assessed for blood glucose levels and then processed to both serum and plasma. Depending on the time point and assay, either all or half of the samples (i.e., 10 or 11) per group were assessed for the following parameters:
Serum: selected serum chemistry parameters [Ala aminotransferase (ALT), Asp aminotransferase (AST), albumin (ALB), and total bile acids (BA)], insulin, and glucagon-like peptides 1 and 2 (GLP-1 and -2)
Plasma: Hemoglobin A1c (HbA1c) and cytokines.

On Day 366 (Week 52), fasted mice were weighed, subjected to terminal cardiocentesis and euthanized. Blood was processed to serum. Livers were excised, weighed, flash-frozen, and stored at −80° C.; remaining tissues were discarded. Serum was assessed for low- and high-density cholesterol (LDL and HDL, respectively) and triglycerides (TGA). Liver was assessed for total cholesterol (TC), TGA, and free fatty acids (FFA). Blood and liver parameters were measured using the kits and equipment indicated in Table 3.

TABLE 3
serum parameter kits and equipment

| Parameter | Matrix | Kit/Equipment Name | Vendor | Catalog No. |
|---|---|---|---|---|
| ALT | Serum | Alanine Aminotransferase Activity | Sigma-Aldrich | MAK052 |
| | | Assay Kit (Sigma-aldrich,) | | |
| AST | Serum | Aspartate Aminotransferase Activity | Sigma-Aldrich | MAK055 |
| | | Assay Kit | | |
| ALB | Serum | Albumin (BCG) Assay Kit | abcam | ab235628 |
| | | (Colorimetric) | | |
| BA | Serum | Mouse Total Bile Acids Kit | Crystal Chem | 80470 |
| Cytokines | Plasma | MILLIPLEX MAP Mouse | Millipore- | MCYTMAG70PMX32BK |
| | | Cytokine/Chemokine Magnetic | Sigma | |
| | | Bead Panel - Premixed 32 Plex - | | |
| | | Immunology Multiplex Assay | | |
| FFA | Liver | Free fatty acid quantitation kit | Sigma-Aldrich | MAK044 |
| GLP-1 | Serum | Mouse GLP-1 ELISA Kit | Crystal Chem | 81508 |
| GLP-2 | Serum | Mouse GLP-2 ELISA Kit | Crystal Chem | 81514 |
| Glucose | Blood | Xx glucometer | Xxx | Xxx |
| | | Xx strips | xxxx | xxxx |
| HbA1c | Plasma | Mouse Hemoglobin A1c Kit | Crystal Chem | 80310 |
| HDL | Serum | Mouse HDL-Cholesterol Kit | Crystal Chem | 79990 |
| Insulin | Serum | Ultra-Sensitive Mouse Insulin | Crystal Chem | 90080 |
| | | ELISA Kit | | |
| LDL | Serum | Mouse LDL-Cholesterol Kit | Crystal Chem | 79980 |
| TC | Liver | Total Cholesterol and Cholesteryl | BioVision | K603-100 |
| | | Ester Colorimetric/Fluorometric | | |
| | | Assay Kit | | |
| TGA | Liver, | Triglyceride Assay Kit | abcam | ab65336 |
| | Serum | | | |

ALB, albumin. ALT, Ala aminotransferase. AST, Asp aminotransferase. BA, bile acids. FFA, free fatty acids. GLP-1/2, glucagon-like peptide-1/2. HDL, high-density lipopeptide. LDL, low-density lipopeptide. No. Number. TC, total cholesterol TGA, triglycerides.

### Results:

For both groups, body weights exhibited a mean increase of approximately 200% (i.e., tripling in weight) from baseline during the year-long study, with the majority of this increase (˜ 150%) occurring during the first 5 months. Notably, body weights (whether expressed as absolute or relative values) did not differ in a statistically significant manner between the two groups during the study, with the sole exception of relative body weights at the first time point (Day 4) after the start of dosing (when relative body weights were lower in the DA-dosed mice of Group 2).

FIG. 1 show group mean absolute (FIG. 1A) and relative (% baseline; FIG. 1B) body weights of AMLN diet-fed mice during one year of daily treatment with vehicle or 30 mg/kg DA.

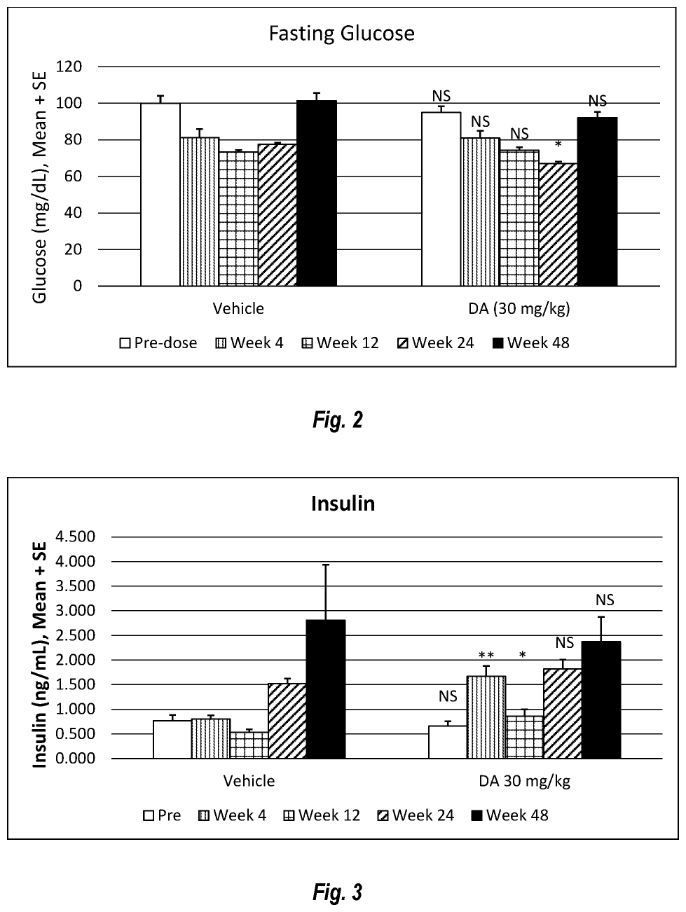

FIG. 2 shows fasting blood glucose levels in the DA-treated mice were significantly lower than those in vehicle-treated mice at Week 24 only. Comparison to vehicle value on respective day by two-tailed non-paired t-test. A difference was considered statistically significant with p<0.05. *p<0.05. NS, not significant.

FIG. 3 shows insulin levels in the DA-treated mice were significantly elevated (compared to those in vehicle-treated mice) at Weeks 4 and 12, but not subsequently. Comparison to vehicle value on respective day by two-tailed non-paired t-test. A difference was considered statistically significant with p<0.05. *p<0.05. **p<0.01. NS, not significant.

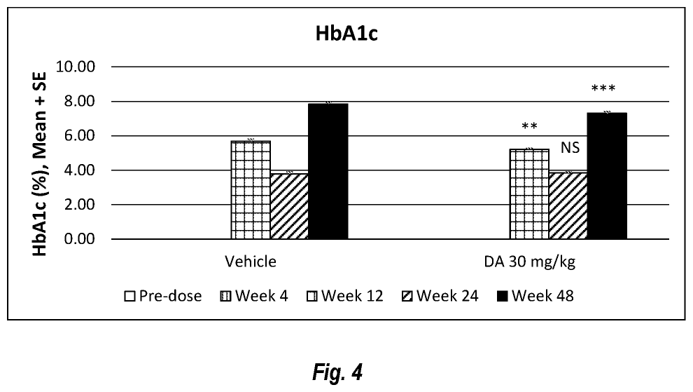

FIG. 4 shows HbA1c levels in the DA-treated mice were significantly attenuated (compared to those in vehicle-treated mice) at Weeks 12 and 48, though not at Week 24. (HbA1c levels were not assayed pre-dose or at Week 4.) Comparison to vehicle value on respective day by two-tailed non-paired t-test. A difference was considered statistically significant with p<0.05. *p<0.05. **p<0.01. ***p<0.001. NS, not significant. HbA1c not assayed pre-dose or at Week 4.

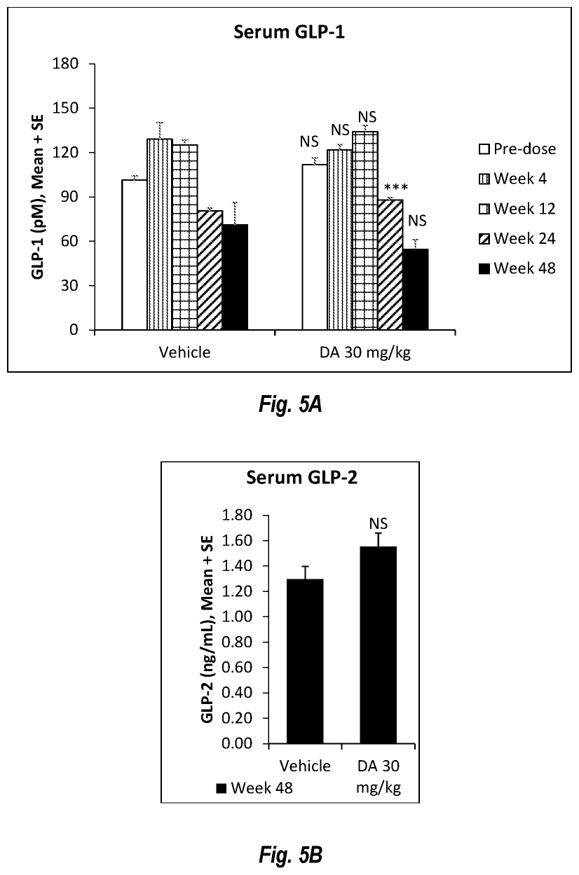

FIGS. 5A-B show GLP-1 levels in the DA-treated mice were significantly attenuated (compared to those in vehicle-treated mice) only at Week 24. While the magnitude of the difference was similar to that at other time points (which lack statistical significance), the variance at Week 24 was lower than at other weeks, suggesting that the statistical significance this effect was not meaningful. Comparison to vehicle value on respective day by two-tailed non-paired t-test. A difference was considered statistically significant with p<0.05. ***p<0.001. NS, not significant. Note that GLP-2 was assayed only at Week 48. GLP-2 levels in the DA-treated mice did not differ significantly from those in vehicle-treated mice at Week 48, the only time point for which this parameter was assessed.

### Serum Chemistries:

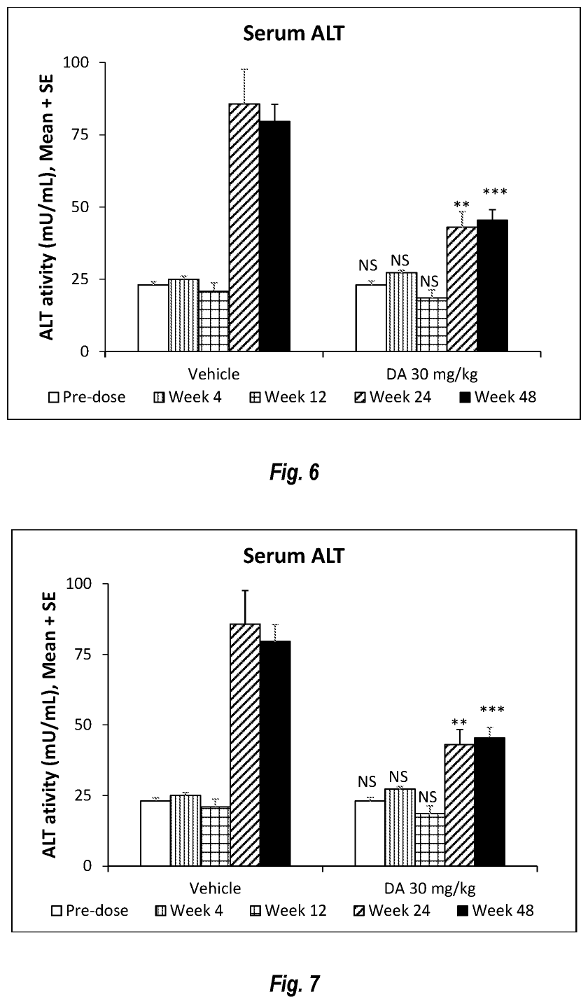

FIG. 6 shows Serum ALT activity levels in the DA-treated mice were significantly attenuated (compared to those in vehicle-treated mice) at Weeks 24 and 48, but not earlier. Comparison to vehicle value on respective day by two-tailed non-paired t-test. A difference was considered statistically significant with p<0.05. **p<0.01. ***p<0.001. NS, not significant.

FIG. 7 shows Serum AST activity levels in the DA-treated mice were significantly attenuated (compared to those in vehicle-treated mice) only at Week 24. Comparison to vehicle value on respective day by two-tailed non-paired t-test. A difference was considered statistically significant with p<0.05. **p<0.01. NS, not significant.

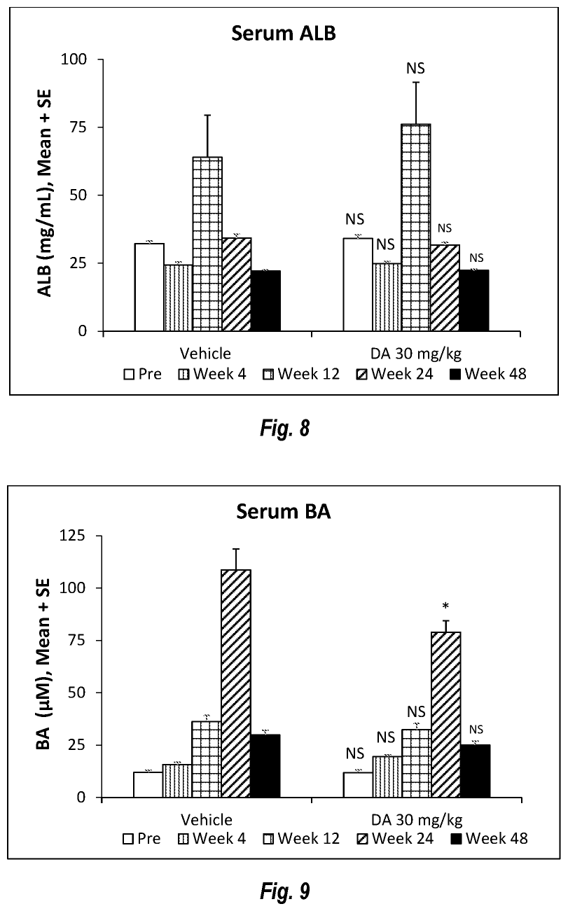

FIG. 8 shows Serum ALB levels in the DA-treated mice did not differ significantly from those in vehicle-treated mice at any assessed time point. Comparison to vehicle value on respective day by two-tailed non-paired t-test. A difference was considered statistically significant with p<0.05. NS, not significant.

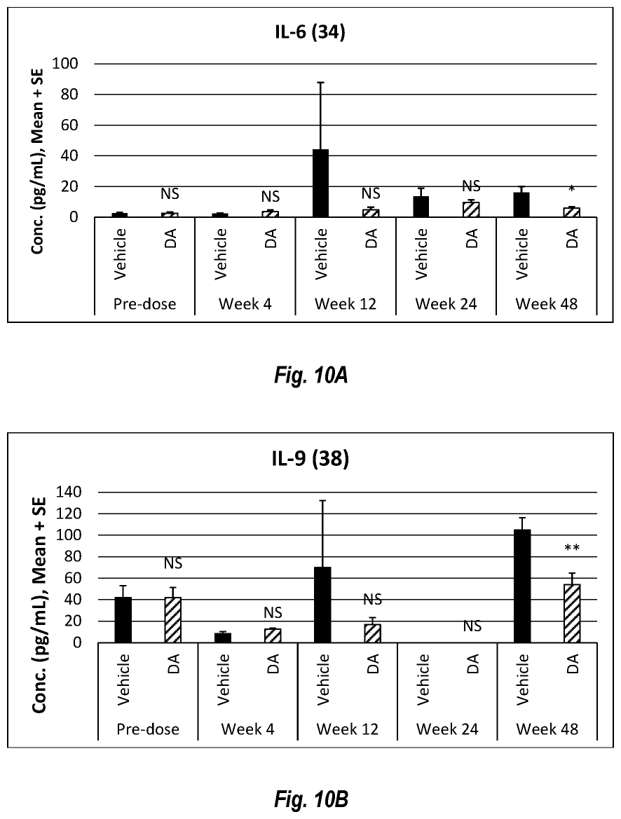

FIG. 9 shows Serum BA levels in the DA-treated mice were significantly attenuated (compared to those in vehicle-treated mice) at Week 24 only. Comparison to vehicle value on respective day by two-tailed non-paired t-test. A difference was considered statistically significant with p<0.05. *p<0.05. NS, not significant.

### Blood Cytokine Levels

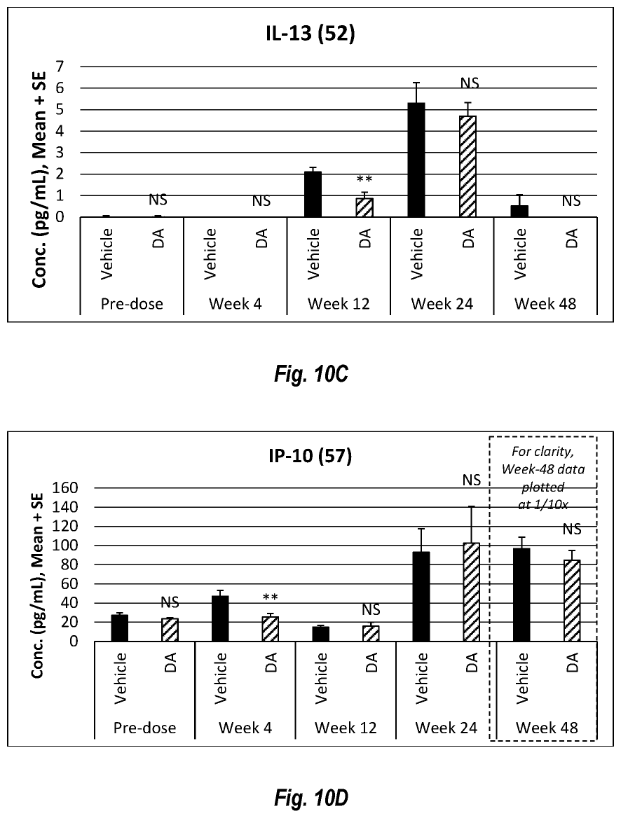

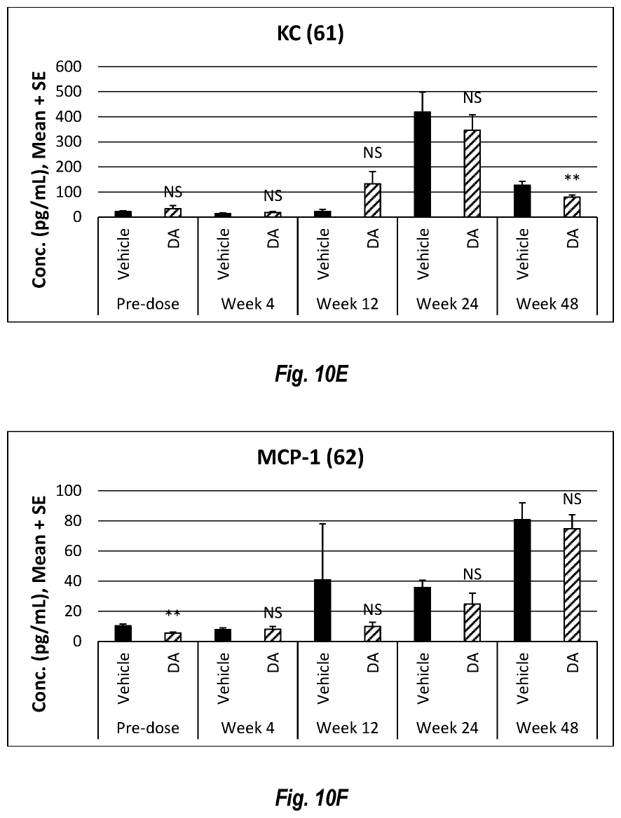

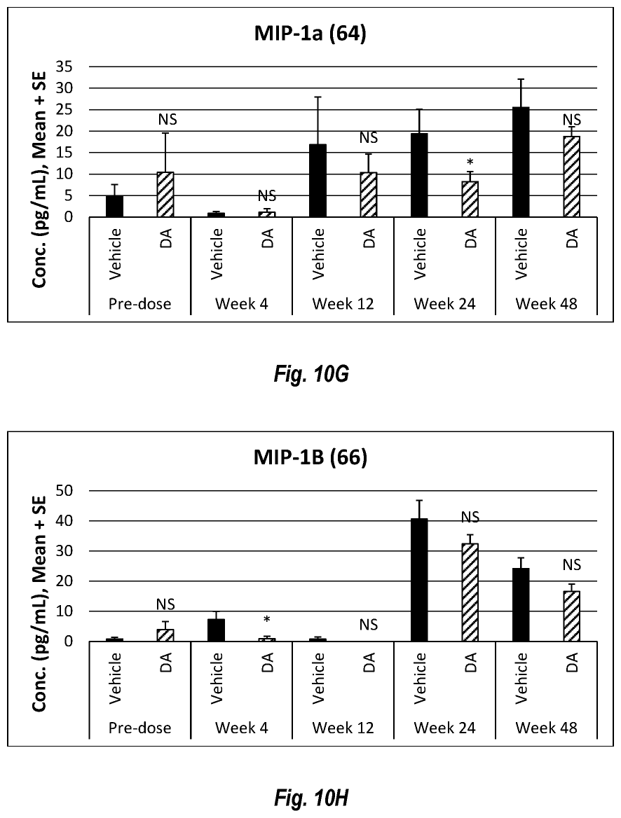

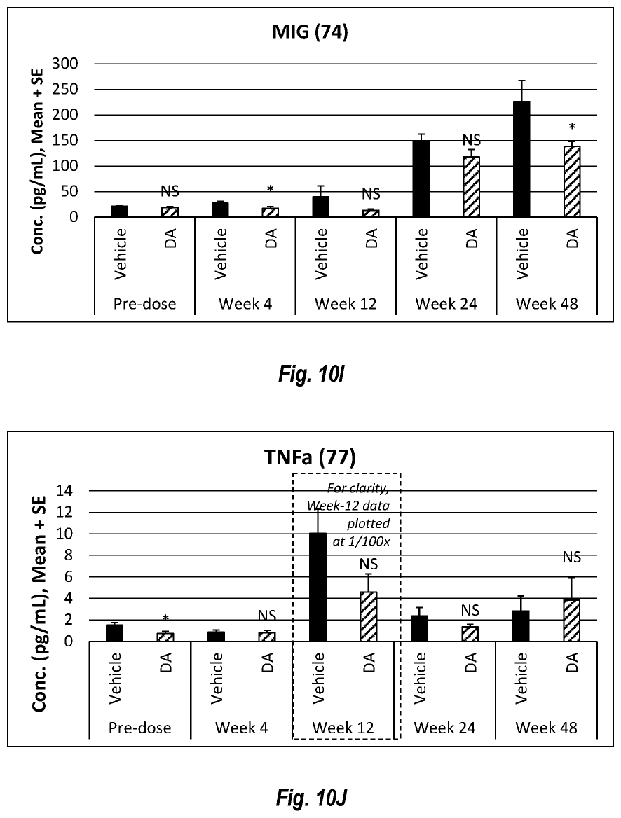

Mean values for cytokines with statistically significant differences between the groups (n=10) are plotted in FIG. 10. Of these 10 cytokines, eight showed significant inter-group differences following the start of dosing. The other two cytokines [MCP-1 (monocyte chemoattractant protein-1 aka CCL2) and TNFα (tumor necrosis factor α)] exhibited significant inter-group differences at the pre-dose time point only, effects that clearly were not related to treatment. Statistically significant attenuation of cytokine levels in the DA-treated mice (compared to vehicle-treated mice) were observed as follows:
Week 4 only: IP-10 (interferon γ-induced protein 10 aka CXCL10); MIP-1B (macrophage inflammatory protein 1β aka CCL4).
Week 12 only: IL-13 (interleukin 13).
Week 24 only: MIP-1a (macrophage inflammatory protein 1α).
Week 48 only: IL-6 (interleukin 6); IL-9 (interleukin 9); KC (keratinocyte-derived chemokine aka CXCL1 or CINC-1).
Weeks 4 and 48 only: MIG (monokine induced by gamma interferon, aka CXCL9).

FIG. 10 shows Blood levels of cytokines (n=10) that showed statistically significant differences in AMLN diet-fed mice during one year of daily treatment with vehicle or 30 mg/kg DA for IL-6 (FIG. 10A), IL-9 (FIG. 10B), IL-13 (FIG. 10C), IP-10 (FIG. 10D), KC (FIG. 10E), MCP-1 (FIG. 10F), MIP-1a (FIG. 10G), MIP-1B (FIG. 10H), MIG (FIG. 10I), and TNFα-6 (FIG. 10J). Parenthetical integers in cytokine names/graph titles correspond to assay numbers used as part of the multiplex assay. Comparison to vehicle value on respective day by two-tailed non-paired t-test. A difference was considered statistically significant with p<0.05. *p<0.05. **p<0.01. NS, not significant.

### Terminal Liver Parameters

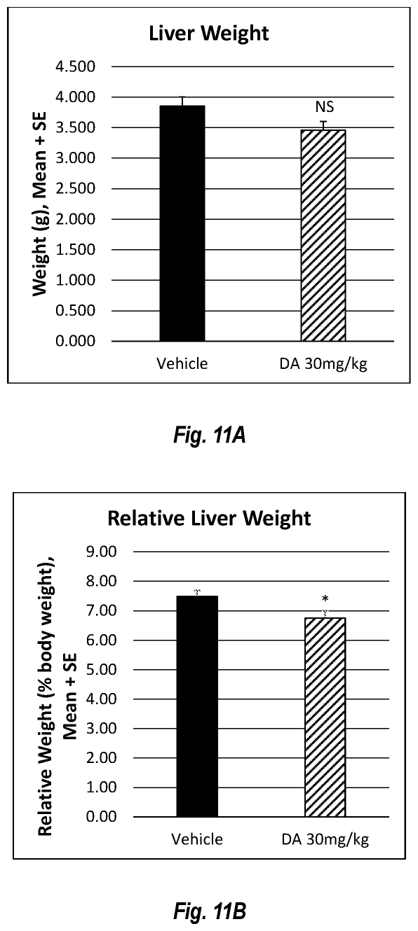

FIGS. 11A (absolute) and B relative (body-weight-normalized) show necropsy liver weight after one year of daily treatment with vehicle of 30 mg/kg DA. Comparison to vehicle value on respective day by two-tailed non-paired t-test. A difference was considered statistically significant with p<0.05. *p<0.05. NS, not significant.

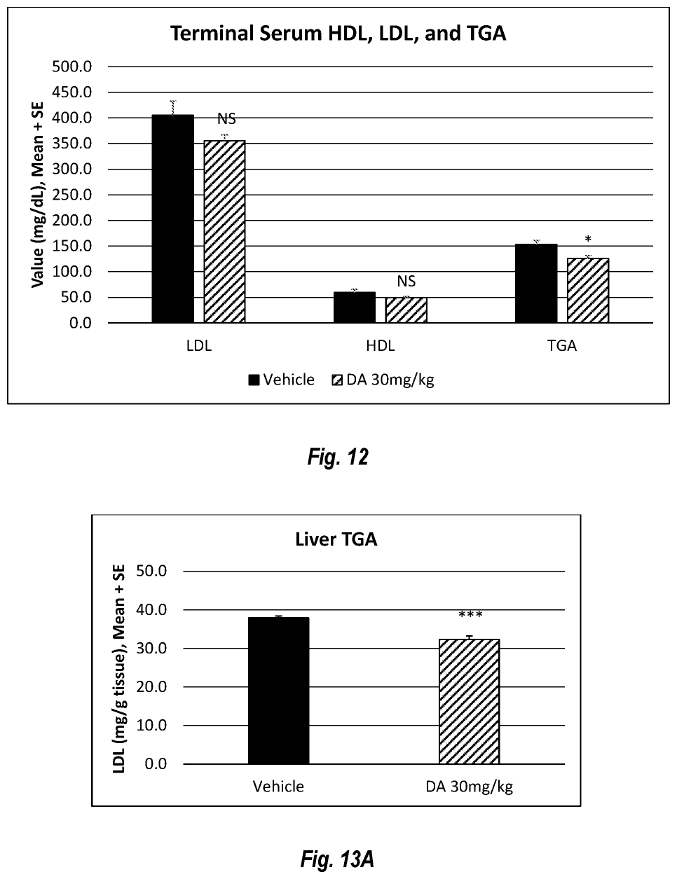

FIG. 12 shows serum HDL, LDL, and TGA levels after one year of daily treatment with vehicle or 30 mg/kg DA. Serum TGA levels, but not those of HDL or LDL, were significantly attenuated in the DA-treated mice (compared to vehicle-dosed animals) after one year of daily dosing. DA, denatonium acetate. HDL, high-density lipo-protein. LDL. Low-density lipoprotein. TGA, triglycerides. Comparison to vehicle value on respective day by two-tailed non-paired t-test. A difference was considered statistically significant with p<0.05. *p<0.05. NS, not significant.

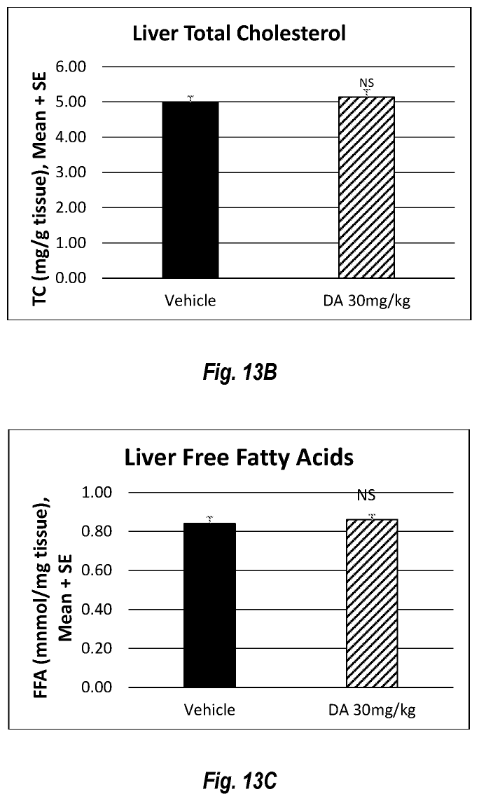

FIGS. 13A-C show liver TGA (FIG. 13A), TC (FIG. 13B) and FFA (FIG. 13 C) after one year of daily treatment with vehicle of 30 mg/kg DA. Liver TGA levels, but not those of TC or FFA, were significantly attenuated in the DA-treated mice (compared to vehicle-dosed mice) after one year of daily dosing.

The conclusion of this study is that one year of DA dosing (30 mg/kg, PO, QD) of AMLN diet-induced NASH mice did not appear to have adverse effects on survival, body weight, or clinical signs. Compared to vehicle, DA resulted in significant changes in a small number of metabolic parameters (fasting glucose, GLP-1, HbA1c, and insulin), serum chemistry (ALT, AST, and BA), and a subset of cytokines (IL-6, IL-9, IL-13, IP-10, KC, MIG, MIP-1a, and MIP-1B) at selected in-life time points. After one year of QD dosing, DA-dosed mice exhibited (compared to vehicle) attenuation of relative liver weight and of TGA levels in both serum and liver.

In conclusion, this one year NASH study histopathology data in FIGS. 12-14 showed that show treatment with DA significantly improved steatosis and improved/abrogated fibrosis (total fibrosis score of 1.52 vs. 0.58 with p-value of <0.0001 as one example) based on blinded histopathologic review. Based on the new FDA NASH/fibrosis criteria, it is the histopathology data, not seen in many other NASH animal studies (often of much shorter duration, see Table 2 herein) support the conclusion that a method for preventing or preventing progression of fatty liver diseases selected from the group consisting of nonalcoholic steatohepatitis (NASH), alcoholic steatohepatitis (ASH), Non-alcoholic fatty liver disease (NAFLD), and HIV-associated steatohepatitis, with or without liver fibrosis, can be accomplished by administering an oral dose of a denatonium salt selected from the group consisting of denatonium acetate (DA), denatonium citrate, denatonium maleate, denatonium saccharide, and denatonium tartrate.

### Example 2

This example provides the results obtained from a second in vivo mouse model of fatty liver disease treatment to investigate the therapeutic effect of DA on the treatment of NASH versus positive control semaglutide (a GLP-1 agonist marketed drug for lowering HbA1c). The differences in this study, from the preventive model in Example 1, were that a higher dose of DA (75 mg/kg) was used versus 30 mg/kg, dosing was twice daily (BID versus QD in the Example 1 study), a positive control semaglutide was used, a different mouse strain (B6 mice) was used, and the animals were already adults (23 weeks old in Example 2 versus 4 weeks old in Example 1) when the study began because the animals were already fed an AMLN diet for 17 weeks prior to the study being initiated. The study dose began at 75 mg/kg BID. However, after two weeks of dosing, it was found that this DA dose was not well tolerated, so it was lowered to 50 mg/kg BID for the remaining 10 weeks of dosing (a total of 12 weeks).

The study included 3 groups of 10 mice each, (A) vehicle control with distilled water by gavage BID, (B) DA by gavage BID, and (C) semaglutide 10 mmol/kg sc QD. Body weights and changes were measured 3× per week. Serum metabolic markers (blood glucose, blood insulin, HbA1c, HDL, LDL triglycerides and bile acids) were measured at beginning of dosing (baseline) and end of study. At the end of the study, histopathology of liver samples and serum levels of inflammatory biomarkers (IL-6, TNFα, CK-18 and TGF-β) were evaluated. Histopathology was performed blindly with a scoring scale according to NAFLD Activity Score and Fibrosis Score according to Table 4.

TABLE 4
Nonalcoholic fatty liver disease activity score and
fibrosis score for histopathological assessment
NAFLD Activity Score

| Score | Steatosis | Lobular inflammation | Ballooning degeneration |
|---|---|---|---|
| 0 | <5% | None | None |
| 1 | 5-33% | <2 foci/20x field | Few |
| 2 | >33-66% | 2-4 foci/20x field | Many |
| 3 | >60% | >4 foci/20x field | |

Fibrosis Score

| Stage | Histological findings |
|---|---|
| 1a | Mild pericellular fibrosis (only seen on connective tissue stain) |
| 1b | Moderate pericellular fibrosis (readily seen on H&E) |
| 1c | Portal/periportal fibrosis without pericellular fibrosis |
| 2 | Pericellular and portal/periportal fibrosis |
| 3 | Bridging fibrosis |
| 4 | Cirrhosis |

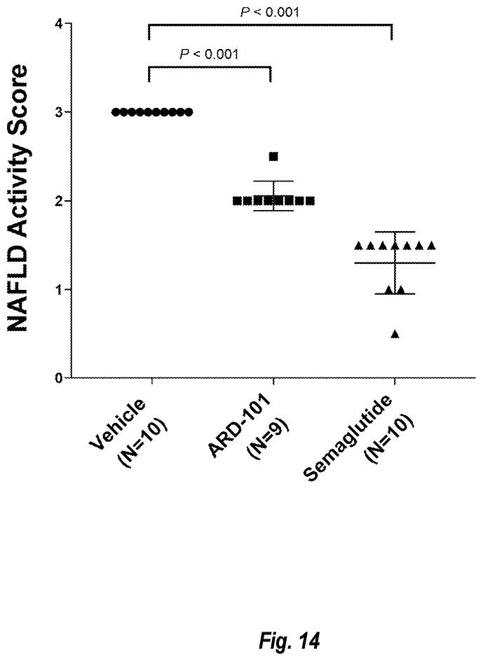

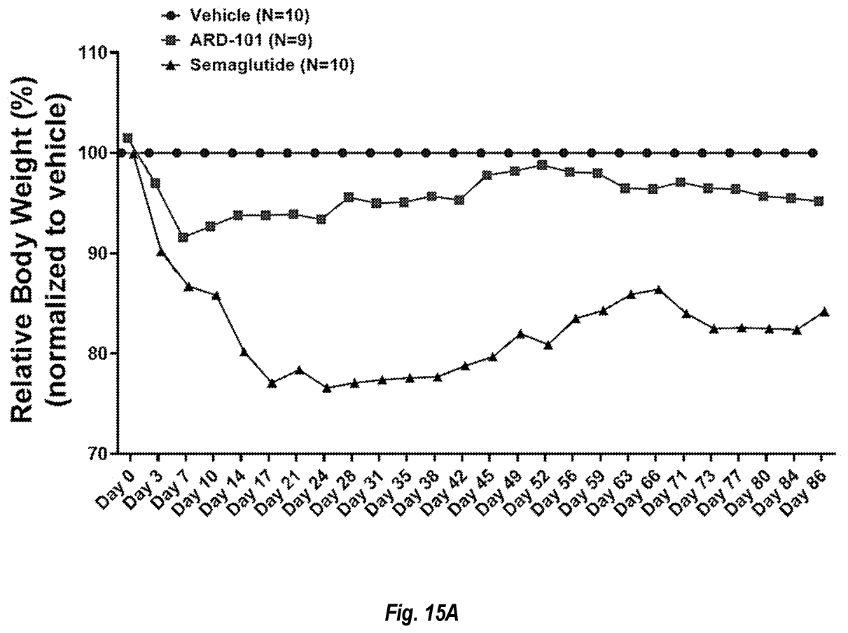

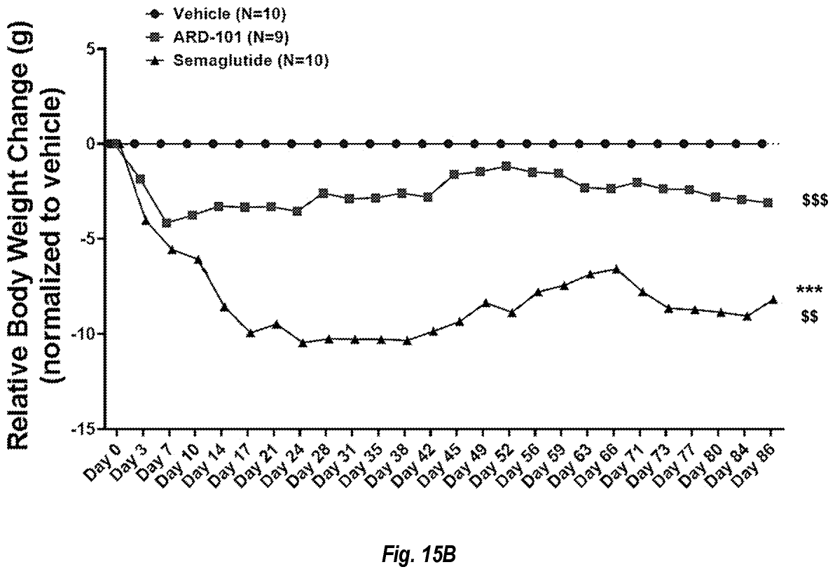

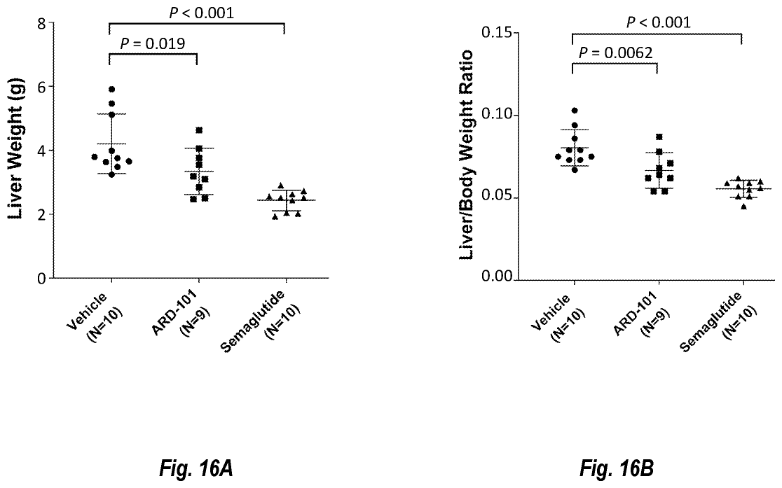

FIG. 14 shows that treatment with DA (ARD-101) significantly improved NAFLD Activity Score based on blinded histopathologic review. FIG. 15A-B shows that treatment with ARD-101 (DA) showed a remarkable effect on body weight and body weight change (FIG. 16A-B) in AMLN-diet induced NASH mice.

FIGS. 16A and 16B show liver weight (FIG. 16A) and liver/body weight ratio (FIG. 16B) showing that DA (ARD-101) significantly decreased liver weight and liver/body weight ratio as compared to vehicle.

FIG. 17A shows ALT levels and FIG. 17B shows AST levels. At the end of the study, DA (ARD-101) significantly decreased ALT and AST levels as compared to vehicle control.

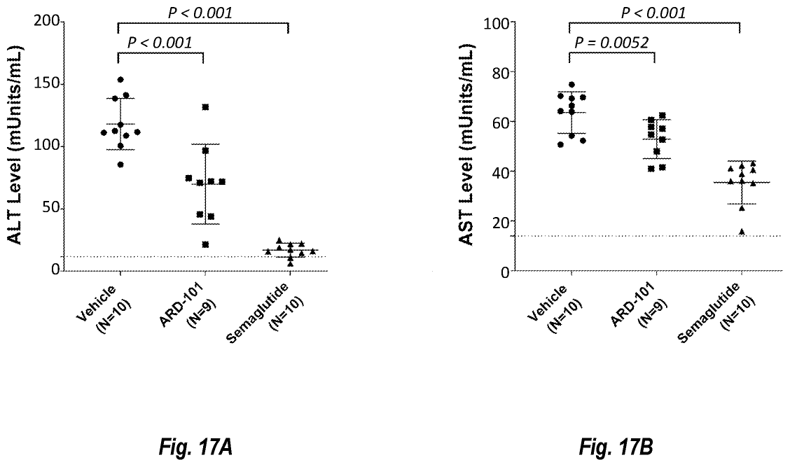

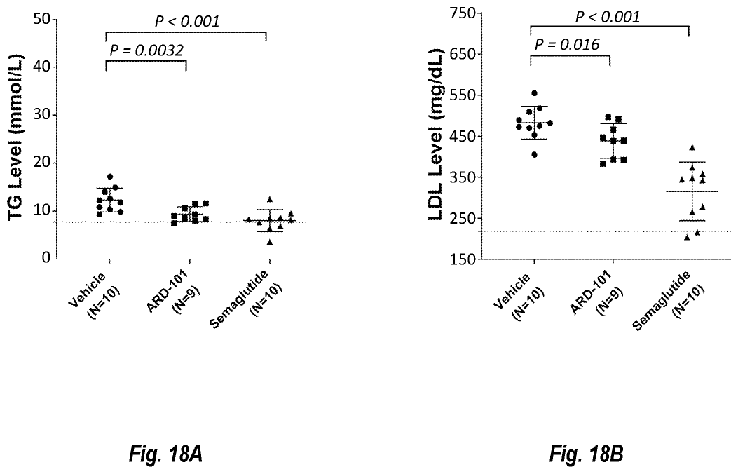

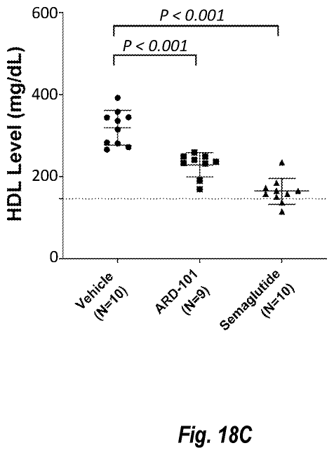

FIGS. 18A (triglycerides), 18B (LDL) and 18C (HDL) show that at the end of the study DA (ARD-101) significantly decreased triglycerides (TG), low density lipoproteins (LDL) and high density lipoproteins (HDL), respectively.

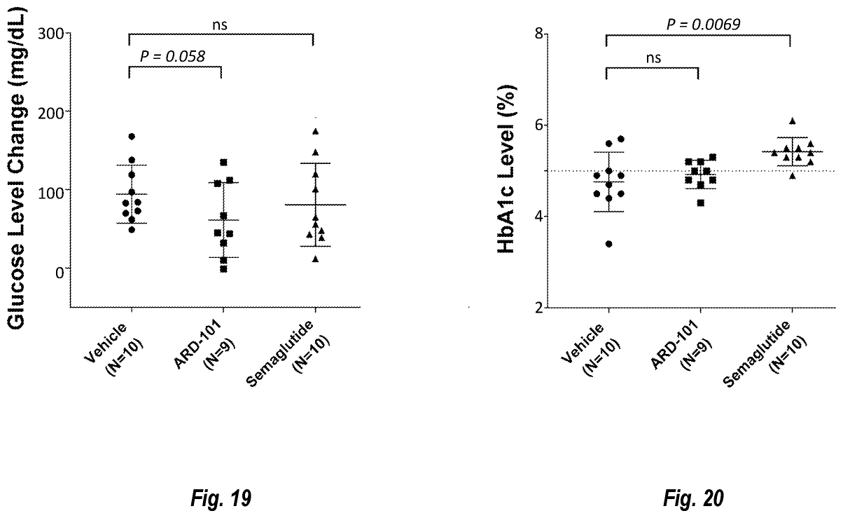

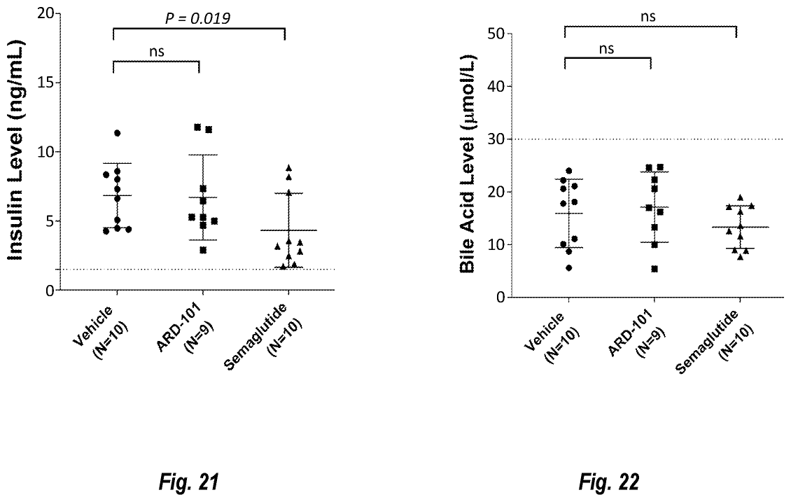

FIG. 19 shows fasting glucose levels at the end of the study. FIG. 20 shows HbA1c levels at the end of the study. The baseline HbA1c level was 5.0%. FIG. 21 shows insulin levels at the end of the study. The baseline insulin level was 1.5 ng/ml. FIG. 22 shows that the two treatments did not significantly impact bile acid levels as compared to vehicle control. The baseline bile acid level was 30 μmol/L.

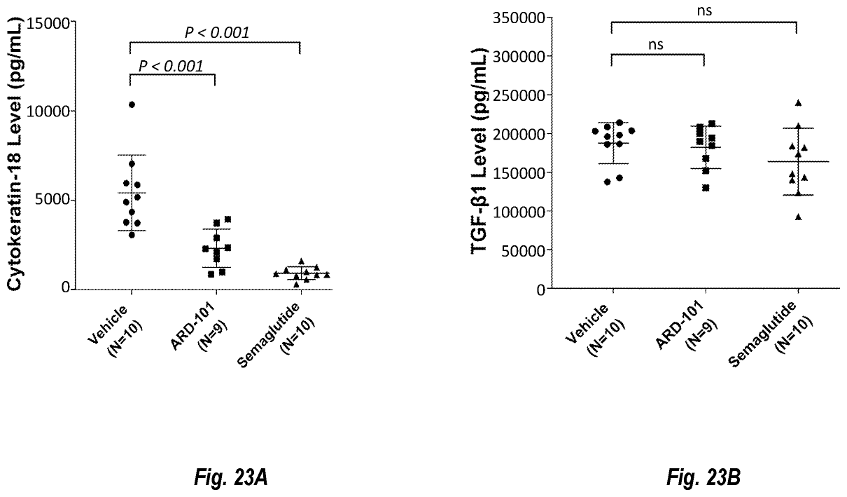

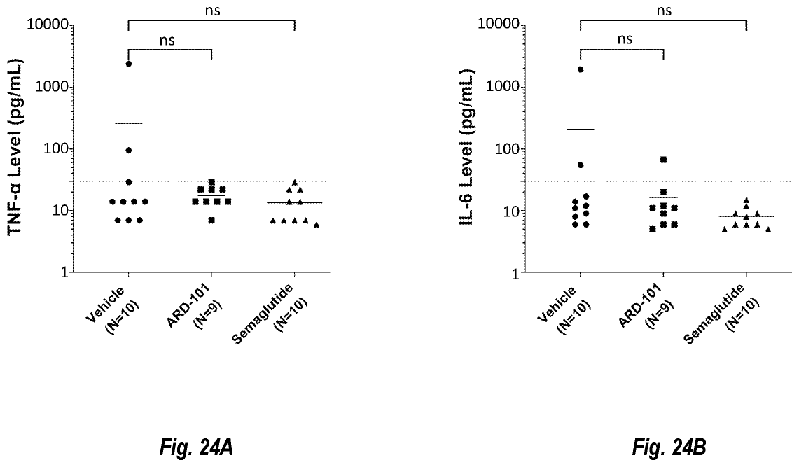

FIGS. 23A (CK-18) and 23B (TGF-β) show that DA (ARD-101) significantly decreased CK-18 levels compared to vehicle control (FIG. 24A).

FIGS. 24A and 24B show that at the end of the study, the two treatments did not significantly impact IL-6 and TNF-α levels as compared to vehicle.

1. A method for preventing, preventing progression, and/or treating a fatty liver disease with or without liver fibrosis, comprising administering an effective amount of a pharmaceutical composition comprising a bitter receptor agonist comprising a denatonium salt, wherein the denatonium salt is selected from the group consisting of denatonium acetate (DA), denatonium citrate, denatonium maleate, denatonium saccharide, and denatonium tartrate.

2. The method of claim 1, wherein the dosage range of the denatonium salt for the method of treatment of nonalcoholic steatohepatitis (NASH) and related liver diseases in a human adult is from about 1.0 mg/kg/day to about 12 mg/kg/day.

3. The method of claim 2, wherein the daily dosage of the denatonium salt for an adult is from about 2 mg/kg/day to about 8 mg/kg/day.

4. The method of claim 3, wherein the daily dosage of DA for an adult is from about 3 mg/kg/day to about 6 mg/kg/day.

5. The method of claim 1, wherein the method is for prevention or preventing progression of the fatty liver disease in a human adult and the dosage range of the denatonium salt is from about 0.1 mg/kg/day to about 8 mg/kg/day, optionally wherein the fatty liver disease comprises NASH.

6. The method of claim 5, wherein the daily dosage of the denatonium salt for an adult is from about 0.25 mg/kg/day to about 4 mg/kg/day.

7. The method of claim 6, wherein the daily dosage of the denatonium salt for an adult is from about 0.5 mg/kg/day to about 3 mg/kg/day.

8. The method of claim 1, wherein the daily dose of the denatonium salt is administered once per day (QD), twice per day (BID) or three times per day (TID).

9. The method of claim 1, wherein the fatty liver disease with or without liver fibrosis is NAFLD, NASH, or ASH.

10. (canceled)

11. (canceled)

12. (canceled)

13. The method of claim 1, wherein the method is for prevention or preventing progression of the fatty liver disease in a human adult and the dosage range of the denatonium salt is from about 0.1 mg/kg/day to about 6 mg/kg/day, optionally wherein the fatty liver disease comprises NASH.

14. (canceled)

15. (canceled)

16. (canceled)

17. The method of claim 1, wherein the fatty liver disease comprises NASH.

18. The method of claim 1, wherein the fatty liver disease comprises ASH.

19. The method of claim 1, wherein the fatty liver disease comprises NAFLD.

20. The method of claim 1, wherein the fatty liver disease comprises human immunodeficiency virus (HIV)-associated steatohepatitis.

21. The method of claim 1, wherein the fatty liver disease includes liver fibrosis.

22. The method of claim 1, wherein the fatty liver disease does not include liver fibrosis.

23. The method of claim 1, wherein the denatonium salt is denatonium citrate.

24. The method of claim 1, wherein the denatonium salt is denatonium tartrate.

25. The method of claim 1, wherein the denatonium salt is denatonium acetate.

26. The method of claim 1, wherein the denatonium salt is denatonium maleate.

27. The method of claim 1, wherein the denatonium salt is denatonium saccharide.
